(19) United States
(12) Patent Application Publication
(10) Pub. No.: US 2026/0130785 A1
(43) Pub. Date: May 14, 2026

(54) SYSTEMS AND METHODS INCLUDING LUBRICANTS FOR CONVEYANCE OF STOOL

(71) Applicant: LIQUIGLIDE, INC., Cambridge, MA (US)

(72) Inventors: Raymond Seekell, Quincy, MA (US); Zheng Zhang, Cambridge, MA (US); Monica Swinney, Stoneham, MA (US); David J. Smith, Newton, MA (US)

(21) Appl. No.: 19/490,118

(22) PCT Filed: Jun. 4, 2024
(86) PCT No.: PCT/US2024/032399

§ 371 Date: Dec. 4, 2025

Related U.S. Application Data
(60) Provisional application No. 63/471,103, filed on Jun. 5, 2023.

(51) Int. Cl. A61F 5/441; A61F 5/44; A61F 5/445; A61L 31/04; A61L 31/06; A61L 31/14; A61L 31/16

(52) U.S. Cl. CPC A61F 5/441; A61F 5/4404; A61F 5/445; A61L 31/048; A61L 31/06; A61L 31/14; A61L 31/16

(57) ABSTRACT

Embodiments described herein relate to lubricating liquids and the application thereof to ostomy pouches. In some embodiments, an apparatus can include a container having an inner surface, and a lubricating liquid coated on the inner surface, between about 50 wt % and about 80 wt % of a primary lubricating oil, between about 0.01 wt % and about 2 wt % of an antioxidant, between about 0.1 wt % and about 10 wt % of a polymer, and between about 1 wt % and about 50 wt % of a secondary liquid, the secondary liquid configured to improve the solubility of the polymer in the primary lubricating oil. In some embodiments, the container includes an ostomy pouch. In some embodiments, the apparatus further includes a contact product disposed in the container and in contact with the lubricating liquid. In some embodiments, the contact product includes stool.

### CROSS REFERENCE TO RELATED APPLICATIONS

This application claims priority to and the benefit of U.S. Provisional Patent Application No. 63/471,103 entitled, “Systems and Methods Including Lubricants for Conveyance of Stool,” filed Jun. 5, 2023, the disclosure of which is incorporated herein by reference in its entirety.

### TECHNICAL FIELD

Embodiments described herein relate to lubricating surfaces and methods of producing the same.

### BACKGROUND

Patients who use ostomy pouches (e.g., for ileostomies, colostomies) experience several issues during the use and management of the pouches. Pouches typically require frequent cleaning and can clog near the stoma (i.e., pancaking), due to a lack of fast, efficient falling of the stool from the stoma area. Deodorizing lubricants in the current state of the art are often difficult or messy to apply and are ineffective if not applied in large quantities. They also do not last more than one cleaning cycle before requiring reapplication, and they are poor at preventing pancaking. Thus, there exists a need for a more durable coating.

### SUMMARY

Embodiments described herein relate to lubricating liquids and the application thereof to ostomy pouches. In some embodiments, an apparatus can include a container having an inner surface, and a lubricating liquid coated on the inner surface, between about 50 wt % and about 90 wt % of a primary lubricating oil, between about 0.01 wt % and about 2 wt % of an antioxidant, between about 0 wt % and about 2 wt % of a deodorant, between about 0.1 wt % and about 10 wt % of a polymer, and between about 1 wt % and about 50 wt % of a secondary liquid, the secondary liquid configured to improve the solubility of the polymer in the primary lubricating oil. In some embodiments, the container includes an ostomy pouch. In some embodiments, the apparatus further includes a contact product disposed in the container and in contact with the lubricating liquid. In some embodiments, the contact product includes stool.

### BRIEF DESCRIPTION OF THE DRAWINGS

FIG. 1 is a block diagram of an apparatus including a lubricating liquid, according to an embodiment.

FIG. 2 is a block diagram of a lubricating liquid, according to an embodiment.

FIGS. 3A-3B are illustrations of an ostomy pouch, according to an embodiment.

FIG. 4 is a flow diagram of a method of producing a lubricated surface, according to an embodiment.

FIGS. 5A-5C are graphical representations comparing Type 3 stool drop times, drain times, and residual mass of ostomy bags with lubricants described herein to commercially available lubricants.

FIGS. 6A-6C are graphical representations comparing Type 6 stool drop times, drain times, and residual mass of ostomy bags with lubricants described herein to commercially available lubricants.

FIGS. 7A-7C are graphical representations comparing Type 3 stool drop times, drain times, and residual mass of ostomy bags with various doses of lubricants described herein.

FIGS. 8A-8C are graphical representations comparing stool drop times, drain times, and residual mass of ostomy bags with various doses of lubricants described herein.

FIG. 9 is a graphical representation of viscosity and stress of a coating described herein as a function of shear rate.

FIG. 10 shows agglomeration of particles in lubricants with various particle concentrations by weight percentage.

FIGS. 11A-11C show contoured interferometer microscope images of lubricants with particles compared to a lubricant without particles. FIG. 11A shows an interferometer microscope image of a lubricant with particles, according to an embodiment. FIG. 11B shows a raw image from the interferometer microscope of a lubricant with particles, according to an embodiment, a processed version of this image with form removal applied, and a simulated photograph based on the image data. FIG. 11C shows three-dimensional views of an interferometer microscope image of a lubricant with particles, and a lubricant without particles.

FIGS. 12A-12C show visual representations of a lubricants' effects on an adhesive ostomy wafer, where FIG. 12A includes lubricants described herein, and FIG. 12B represents aqueous-based lubricants.

FIG. 13 demonstrates a pouch tail squeeze method of removing stool from ostomy bags and residual stool mass remaining thereafter.

### DETAILED DESCRIPTION

Lubricating liquids in ostomy pouches in the current state of the art have several defects. Mineral oils, olive oils, cooking sprays, and other commercial lubricating deodorants experience many of the aforementioned issues. Challenges in the application of lubricants for ostomy pouches include variability in stool consistency, stability of lubricant on interior surface of pouch, and maintaining lubricity through multiple cleanings.

Human stools vary considerably in shape, consistency, pH, and water content, making design of ostomy pouches difficult. Medical professionals have developed the Bristol stool scale to classify the form of human feces into seven categories. It is used in both clinical and experimental fields. Type 1 includes separate, hard lumps, with a consistency like nuts (difficult to pass). Type 2 includes sausage-shaped stools with lumps. Type 3 includes sausage-shaped stools with cracks on the surface. Type 4 includes a sausage- or snake-shaped stool (average stool). Type 5 includes soft blobs with clear cut edges. Type 6 includes fluffy pieces with ragged edges, or a mushy stool (diarrhea). Type 7 includes a watery stool with no solid pieces, or entirely liquid (diarrhea).

Stool type is dependent on several variables of food and fluid intake, but Type 3 is common for patients with a colostomy, while Type 6 is common for patients with an ileostomy. Engineering a lubrication system that accommodates a broad range of Bristol scale consistencies is difficult. Embodiments described herein can be used both for patients with colostomies (i.e., Type 6 stools) and all the way through the Bristol scale to ileostomy patients (i.e., Type 3 stools). Embodiments described herein show significant improvement in contact with thicker, stickier stool types.

Furthermore, coatings described herein can retain effectiveness during multiple stool outputs without the need to reapply. Embodiments described herein can last more than 3 stool outputs per day (common for colostomy patients) or more than 6 stools per day (common for ileostomy patients).

Existing lubricants have many shortcomings. Oil-based lubricants used in the current state of the art are not compatible with preferred pouch materials (including ethyl vinyl acetate (EVA)). For example, mineral oil can plasticize the ostomy pouch and weaken the seals of the pouch, compromising integrity. Water-based lubricants reduce adhesion of the pouch wafer and cause degradation of the pouch wafer. Existing lubricants use a large amount of lubricating deodorant, which can make the lubricant messy to apply. Existing lubricants are also not very durable and become damaged or thinned easily.

Incorporation of a polymer (e.g., Cellulose or cellulose derivatives, such as carboxymethyl cellulose (CMC), hydroxyethylcellulose (HEC), hydroxy propylcellulose (HPC), hydroxypropylmethylcellulose (HPMC), methylcellulose (MC), hydroxymethylcellulose (HMC), ethyl methyl cellulose, (MEC), ethyl cellulose (EC)) into an oil (e.g., a vegetable oil or vegetable oil derivative) can modify the rheological properties of the oil, to improve its lubricating properties and stability. The modified viscosity helps the lubricant to remain stable after being applied to the interior of a pouch. The polymer can also impart shear-thinning properties to the lubricant that allows the lubricant to adhere to the interior of the ostomy pouch and then allow sliding when in contact with a stool.

In some embodiments, the lubricating liquid can preferentially wet the interior surface of the pouch and remain stable beneath the stool, that is, the receding contact angle of the liquid on the interior surface materials, with an external phase of air or stool, should be less than about 30°. It is even more preferable if the receding contact is less than about 20°, or less than about 10°. In some embodiments, the lubricating liquid can be stabilized on the interior of the pouch by the polymeric structure and other structural features (e.g., particles), capillarity, as well as selection of materials that provide desirable surface energy. Embodiments described herein can provide significant improvements in stool drop time and non-adhesion of stool to the target surface (e.g., the interior of ostomy pouches). This can lead to a reduced incidence of pancaking and ease of cleaning. Additionally, stability of the lubricating liquid on the surface beneath the stool reduces the need for replenishment, improving the patient's experience and ease of use. The inclusion of a deodorant in the lubricating liquid is also important for the user experience.

In some embodiments, the particles in the lubricant can retain the separation of the walls of an ostomy pouch, causing the gap created between the walls to retain lubricating liquid. The size of such “bridging particles” can be selected to adjust the thickness of the of lubricant coating on the pouch surface when stool enters the pouch. For example, particles or aggregates of particles with average dimension of about 20 μm can retain up to about 20 μm of lubricant between the pouch walls, leavings approximately 10 μm of lubricant on each side after the walls of the pouch are separated by stool entering the pouch. Another important consequence of this separation is that the volume of the lubricant that is held in this space will not drain from the pouch. Accordingly, pouches can be coated with lubricant during the manufacturing process and/or by the pouch manufacturer, without concerns of lubricant draining out of the tail or other opening of the pouch during shipping and handling. In some cases, it is desirable to incorporate bridging particles in the lubricant with an average dimension of about 2 μm, about 5 μm, about 10 μm, about 20 μm, about 30 μm, about 40 μm, or about 50 μm, inclusive of all values and ranges therebetween. In some cases, it is desirable that particles in the lubricant form aggregates with average aggregate dimension of about 2 μm, about 5 μm, about 10 μm, about 20 μm, about 30 μm, about 40 μm, or about 50 μm, inclusive of all values and ranges therebetween. In some embodiments, bridging particles can be included in the lubricant in concentrations of about 0.5 wt %, about 1 wt %, about 5 wt %, about 10 wt %, about 20 wt %, about 50 wt %, inclusive of all values and ranges therebetween. To retain the performance of the lubricant through multiple uses (multiple stool outputs), it is preferable that the bridging particle material be preferentially wetted by lubricating liquid, that is, the receding contact angle of the lubricating liquid on the particle, as measured with an external phase of air or stool, should be less than about 30°. In some embodiments, the receding contact angle can be less than about 20° or less than about 30°. This low receding contact angle minimizes the extent to which the particles are pulled away from the surface by stool passing over them.

Lubricating liquids and lubricating systems described herein can have any of the properties described in International Patent Publication No. WO2020/214684 (“the '684 Publication”), filed Apr. 15, 2020 and titled, “Lubricious Surfaces, Systems and Methods for Making the Same,” the disclosure of which is hereby incorporated by reference in its entirety. Lubricating liquids and lubricating systems described herein can have any of the properties described in International Patent Publication No. WO2018/191523 (“the '523 Publication”), filed Apr. 12, 2018 and titled, “Durable Lubricious Surfaces,” the disclosure of which is hereby incorporated by reference in its entirety.

As used in this specification, the singular forms “a,” “an,” and “the” include plural referents unless the context clearly dictates otherwise. Thus, for example, the term “a member” is intended to mean a single member or a combination of members, “a material” is intended to mean one or more materials, or a combination thereof.

The term “substantially” when used in connection with “cylindrical,” “linear,” and/or other geometric relationships is intended to convey that the structure so defined is nominally cylindrical, linear or the like. As one example, a portion of a support member that is described as being “substantially linear” is intended to convey that, although linearity of the portion is desirable, some non-linearity can occur in a “substantially linear” portion. Such non-linearity can result from manufacturing tolerances, or other practical considerations (such as, for example, the pressure or force applied to the support member). Thus, a geometric construction modified by the term “substantially” includes such geometric properties within a tolerance of plus or minus 5% of the stated geometric construction. For example, a “substantially linear” portion is a portion that defines an axis or center line that is within plus or minus 5% of being linear.

As used herein, the term “set” and “plurality” can refer to multiple features or a singular feature with multiple parts. For example, when referring to a set of electrodes, the set of electrodes can be considered as one electrode with multiple portions, or the set of electrodes can be considered as multiple, distinct electrodes. Additionally, for example, when referring to a plurality of apparatuses, the plurality of apparatuses can be considered as multiple, distinct apparatuses or as one apparatus with multiple portions. Thus, a set of portions or a plurality of portions may include multiple portions that are either continuous or discontinuous from each other. A plurality of particles or a plurality of materials can also be fabricated from multiple items that are produced separately and are later joined together (e.g., via mixing, an adhesive, or any suitable method).

As used herein, the term “about” and “approximately” generally mean plus or minus 10% of the value stated, for example about 250 μm would include 225 μm to 275 μm, about 1,000 μm would include 900 μm to 1,100 μm.

As used herein, the term “contact liquid”, “bulk material,” “contact product,” and “product” are used interchangeably to refer to a solid or liquid that flows, for example a non-Newtonian fluid, a Bingham fluid, a high viscosity fluid, multiphase complex fluid, or a thixotropic fluid and is in contact with a liquid-encapsulated surface and/or lubricating liquid, unless otherwise stated.

As used herein, “particle size” refers to an average distance across a 3-dimensional particle. For a spherical particle, an average dimension would refer to the spherical particle's diameter. For an irregularly shaped particle, the average dimension would refer to the average distance across the particle across all imaginary lines running through the center of mass of the particle.

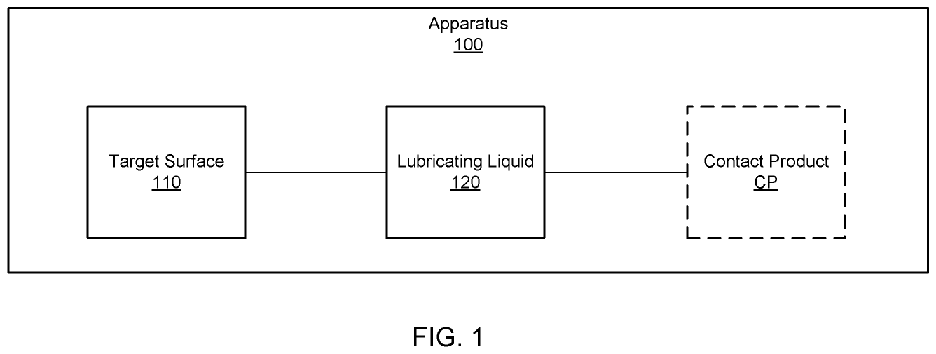

FIG. 1 is a block diagram of an apparatus 100 including a lubricating liquid 120, according to an embodiment. As shown, the lubricating liquid 120 is in contact with a target surface 110 and optionally with a contact product CP.

The target surface 110 is the site of the application of the lubricating liquid 120. In some embodiments, the target surface 110 can include an interior of an ostomy pouch, a commode chair, a potty training product (e.g., a miniature toddler toilet or a toddler toilet seat), a bedpan, a camper toilet, a waterless toilet, a space toilet, a fecal catheter, a surgical bulb, a stent, a pet waste collection scoop (i.e., “pooper scooper”), a pet waste collection bag, and/or a catheter. In some embodiments, the lubricating liquid 120 can be added to a gap or opening in an ostomy wafer. The lubricating liquid 120 can exhibit minimal degradation of the ostomy wafer while in contact with the ostomy wafer. In some embodiments, the target surface 110 can be composed of EVA, polyvinyl chloride (PVC), polyethylene terephthalate (PET), polyurethane, polyethylene, polypropylene, and/or silicone.

In some embodiments, the target surface 110 can stabilize the lubricating liquid 120 via surface features on the target surface. In some embodiments, the surface features can include bumps, ridges, capillaries, and/or particles disposed on or integrated into the target surface 110. In some embodiments, features on the target surface 110 can have a density of about 1 capillary/cm², about 2 features/cm², about 3 features/cm², about 4 features/cm², about 5 features/cm², about 6 features/cm², about 7 features/cm², about 8 features/cm², about 9 features/cm², about 10 features/cm², about 20 features/cm², about 30 features/cm², about 40 features/cm², about 50 features/cm², about 60 features/cm², about 70 features/cm², about 80 features/cm², about 90 features/cm², about 100 features/cm², about 200 features/cm², about 300 features/cm², about 400 features/cm², about 500 features/cm², about 600 features/cm², about 700 features/cm², about 800 features/cm², about 900 features/cm², about 1,000 features/cm², about 2,000 features/cm², about 3,000 features/cm², about 4,000 features/cm², about 5,000 features/cm², about 6,000 features/cm², about 7,000 features/cm², about 8,000 features/cm², about 9,000 features/cm², or about 10,000 features/cm², inclusive of all values and ranges therebetween.

The lubricating liquid 120 is disposed on the target surface 110 and provides a slippery surface. The contact product CP slides off the target surface 110 because of the lubricating properties of the lubricating liquid 120. In some embodiments, the lubricating liquid 120 can include a primary lubricating oil, a secondary liquid, an antioxidant, and a polymer. In some embodiments, the lubricating liquid 120 can include a deodorant. In some embodiments, the deodorant can include chlorophyllin copper, copper citrate, copper gluconate, N-soya-N-ethyl morpholinium ethosulfate (Forestall™), vegetable protein extracts (including but not limited to sodium caproyl/lauroyl lactylate, sodium decanoyl/dodecanoyl lactylate), triethyl citrate), essential oils (including but not limited to pine oil, sage oil, eucalyptus oil, Melaleuca armillaris, peppermint oil, and/or lavender oil), or any combination thereof.

In some embodiments, at least about 0.1 mL, at least about 0.2 mL, at least about 0.3 mL, at least about 0.4 mL, at least about 0.5 mL, at least about 0.6 mL, at least about 0.7 mL, at least about 0.8 mL, at least about 0.9 mL, at least about 1 mL, at least about 2 mL, at least about 3 mL, at least about 4 mL, at least about 5 mL, at least about 6 mL, at least about 7 mL, at least about 8 mL, at least about 9 mL, at least about 10 mL, at least about 20 mL, at least about 30 mL, at least about 40 mL, at least about 50 mL, at least about 60 mL, at least about 70 mL, at least about 80 mL, or at least about 90 mL of the lubricating liquid 120 can be disposed on the target surface 110. In some embodiments, no more than about 100 mL, no more than about 90 mL, no more than about 80 mL, no more than about 70 mL, no more than about 60 mL, no more than about 50 mL, no more than about 40 mL, no more than about 30 mL, no more than about 20 mL, no more than about 10 mL, no more than about 9 mL, no more than about 8 mL, no more than about 7 mL, no more than about 6 mL, no more than about 5 mL, no more than about 4 mL, no more than about 3 mL, no more than about 2 mL, no more than about 1 mL, no more than about 0.9 mL, no more than about 0.8 mL, no more than about 0.7 mL, no more than about 0.6 mL, no more than about 0.5 mL, no more than about 0.4 mL, no more than about 0.3 mL, or no more than about 0.2 mL of the lubricating liquid 120 can be disposed on the target surface 110. Combinations of the above-referenced amounts of lubricating liquid 120 on the target surface 110 are also possible (e.g., at least about 0.1 mL and no more than about 100 mL or at least about 1 mL and no more than about 10 mL), inclusive of all values and ranges therebetween. In some embodiments, about 0.1 mL, about 0.2 mL, about 0.3 mL, about 0.4 mL, about 0.5 mL, about 0.6 mL, about 0.7 mL, about 0.8 mL, about 0.9 mL, about 1 mL, about 2 mL, about 3 mL, about 4 mL, about 5 mL, about 6 mL, about 7 mL, about 8 mL, about 9 mL, about 10 mL, about 20 mL, about 30 mL, about 40 mL, about 50 mL, about 60 mL, about 70 mL, about 80 mL, about 90 mL, or about 100 mL of the lubricating liquid 120 can be disposed on the target surface 110.

The contact product CP slides off of the target surface 110 via the lubricating liquid 120. In some embodiments, the contact product CP can include stool. In some embodiments, the contact product CP can include human stool. In some embodiments, the contact product CP can include animal stool (e.g., dog stool, cat stool, livestock manure). In some embodiments, the contact product CP can have a Bristol scale value of about 1, about 2, about 3, about 4, about 5, about 6, or about 7, inclusive of all values and ranges therebetween.

In some embodiments, the contact product CP can have a viscosity at 20° C. of at least about 100 Pa·s, at least about 150 Pa·s, at least about 200 Pa·s, at least about 250 Pa·s, at least about 300 Pa·s, at least about 350 Pa·s, at least about 400 Pa·s, at least about 450 Pa·s, at least about 500 Pa·s, at least about 550 Pa·s, at least about 600 Pa·s, at least about 650 Pa·s, at least about 700 Pa·s, at least about 750 Pa·s, at least about 800 Pa·s, at least about 850 Pa·s, at least about 900 Pa·s, or at least about 950 Pa·s. In some embodiments, the contact product CP can have a viscosity at 20° C. of no more than about 1,000 Pa·s, no more than about 950 Pa·s, no more than about 900 Pa·s, no more than about 850 Pa·s, no more than about 800 Pa·s, no more than about 750 Pa·s, no more than about 700 Pa·s, no more than about 650 Pa·s, no more than about 600 Pa·s, no more than about 550 Pa·s, no more than about 500 Pa·s, no more than about 450 Pa·s, no more than about 400 Pa·s, no more than about 350 Pa·s, no more than about 300 Pa·s, no more than about 250 Pa·s, no more than about 200 Pa·s, or no more than about 150 Pa·s. Combinations of the above-referenced viscosity values are also possible (e.g., at least about 100 Pa·s and no more than about 1,000 Pa·s or at least about 300 Pa·s and no more than about 700 Pa·s), inclusive of all values and ranges therebetween. In some embodiments, the contact product CP can have a viscosity at 20° C. of about 100 Pa·s, about 150 Pa·s, about 200 Pa·s, about 250 Pa·s, about 300 Pa·s, about 350 Pa·s, about 400 Pa·s, about 450 Pa·s, about 500 Pa·s, about 550 Pa·s, about 600 Pa·s, about 650 Pa·s, about 700 Pa·s, about 750 Pa·s, about 800 Pa·s, about 850 Pa·s, about 900 Pa·s, about 950 Pa·s, or about 1,000 Pa·s.

In some embodiments, the contact product CP can have a yield stress at 20° C. of at least about 1 Pa, at least about 2 Pa, at least about 3 Pa, at least about 4 Pa, at least about 5 Pa, at least about 6 Pa, at least about 7 Pa, at least about 8 Pa, at least about 9 Pa, at least about 10 Pa, at least about 20 Pa, at least about 30 Pa, at least about 40 Pa, at least about 50 Pa, at least about 60 Pa, at least about 70 Pa, at least about 80 Pa, at least about 90 Pa, at least about 100 Pa, at least about 110 Pa, at least about 120 Pa, at least about 130 Pa, at least about 140 Pa, at least about 150 Pa, at least about 160 Pa, at least about 170 Pa, at least about 180 Pa, at least about 190 Pa, at least about 200 Pa, at least about 210 Pa, at least about 220 Pa, at least about 230 Pa, at least about 240 Pa, at least about 250 Pa, at least about 260 Pa, at least about 270 Pa, at least about 280 Pa, or at least about 290 Pa. In some embodiments, the contact product CP can have a yield stress at 20° C. of no more than about 300 Pa, no more than about 290 Pa, no more than about 280 Pa, no more than about 270 Pa, no more than about 260 Pa, no more than about 250 Pa, no more than about 240 Pa, no more than about 230 Pa, no more than about 220 Pa, no more than about 210 Pa, no more than about 200 Pa, no more than about 190 Pa, no more than about 180 Pa, no more than about 170 Pa, no more than about 160 Pa, no more than about 150 Pa, no more than about 140 Pa, no more than about 130 Pa, no more than about 120 Pa, no more than about 110 Pa, no more than about 100 Pa, no more than about 90 Pa, no more than about 80 Pa, no more than about 70 Pa, no more than about 60 Pa, no more than about 50 Pa, no more than about 40 Pa, no more than about 30 Pa, no more than about 20 Pa, no more than about 10 Pa, no more than about 9 Pa, no more than about 8 Pa, no more than about 7 Pa, no more than about 6 Pa, no more than about 5 Pa, no more than about 4 Pa, no more than about 3 Pa, or no more than about 2 Pa. Combinations of the above-referenced yield stress values are also possible (e.g., at least about 1 Pa and no more than about 300 Pa or at least about 10 Pa and no more than about 200 Pa), inclusive of all values and ranges therebetween. In some embodiments, the contact product CP can have a yield stress at 20° C. of about 1 Pa, about 2 Pa, about 3 Pa, about 4 Pa, about 5 Pa, about 6 Pa, about 7 Pa, about 8 Pa, about 9 Pa, about 10 Pa, about 20 Pa, about 30 Pa, about 40 Pa, about 50 Pa, about 60 Pa, about 70 Pa, about 80 Pa, about 90 Pa, about 100 Pa, about 110 Pa, about 120 Pa, about 130 Pa, about 140 Pa, about 150 Pa, about 160 Pa, about 170 Pa, about 180 Pa, about 190 Pa, about 200 Pa, about 210 Pa, about 220 Pa, about 230 Pa, about 240 Pa, about 250 Pa, about 260 Pa, about 270 Pa, about 280 Pa, about 290 Pa, or about 300 Pa.

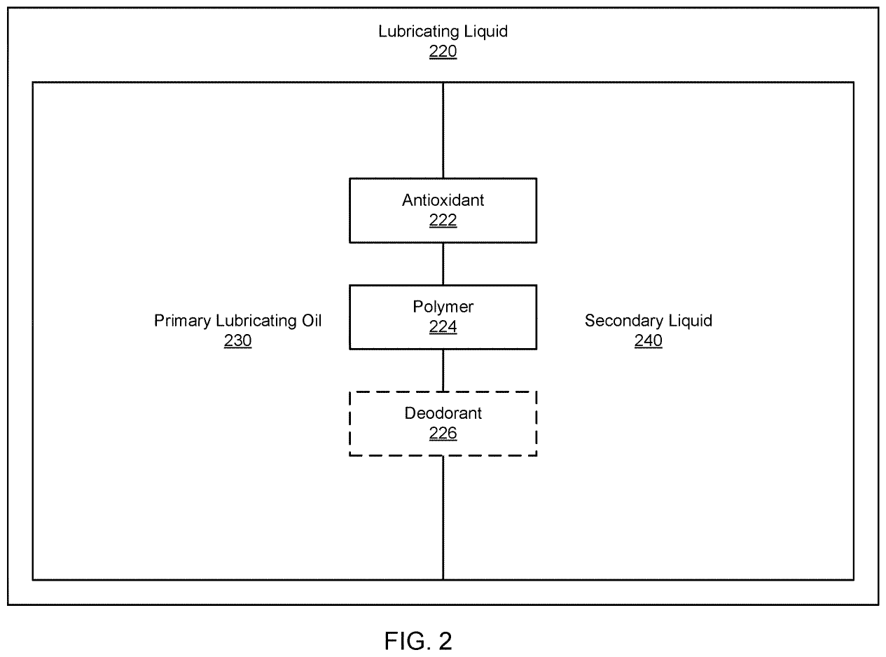

FIG. 2 is a block diagram of a lubricating liquid 220, according to an embodiment. As shown, the lubricating liquid 220 a primary lubricating oil 230 and a secondary liquid 240 with an antioxidant 222, a polymer 224, and optionally a deodorant 226 included in the lubricating liquid 220. In some embodiments, the lubricating liquid 220 can include a solution, a suspension, a mixture, and/or a colloid.

The primary lubricating oil 230 provides a slippery or base material for the lubricating liquid 220. In some embodiments, the primary lubricating oil 230 can make up at least about 50 wt %, at least about 55 wt %, at least about 60 wt %, at least about 65 wt %, at least about 70 wt %, at least about 75 wt %, at least about 80 wt %, at least about 85 wt %, at least about 90 wt %, at least about 95 wt %, at least about 96 wt %, at least about 97 wt %, or at least about 98 wt % of the lubricating liquid 220. In some embodiments, the primary lubricating oil 230 can make up no more than about 99 wt %, no more than about 98 wt %, no more than about 97 wt %, no more than about 96 wt %, no more than about 95 wt %, no more than about 90 wt %, no more than about 85 wt %, no more than about 80 wt %, no more than about 75 wt %, no more than about 70 wt %, no more than about 65 wt %, no more than about 60 wt %, or no more than about 55 wt % of the lubricating liquid 220. Combinations of the above-referenced composition percentages are also possible (e.g., at least about 50 wt % and no more than about 90 wt % or at least about 60 wt % and no more than about 80 wt %), inclusive of all values and ranges therebetween. In some embodiments, the primary lubricating oil 230 can make up about 50 wt %, about 55 wt %, about 60 wt %, about 65 wt %, about 70 wt %, about 75 wt %, about 80 wt %, about 85 wt %, about 90 wt %, about 95 wt %, about 96 wt %, about 97 wt %, about 98 wt %, or about 99 wt % of the lubricating liquid 220.

In some embodiments, the primary lubricating oil 230 can include a vegetable oil, a vegetable oil derivative, a mineral oil, a synthetic liquid, a polyolefin, a hydrogenated polyolefin, an ester, a silicone, a fluorocarbon, a genetically modified vegetable oil, and/or a fractionated vegetable oil. In some embodiments, the vegetable oil can include a natural vegetable oil, a soybean oil, an olive oil, a cottonseed oil, a synthetic vegetable oil, and/or a genetically modified vegetable oil.

The secondary liquid 240 is mixed with the primary lubricating oil 230. In some embodiments, the secondary liquid 240 can be fully mixed with the primary lubricating oil 230. In some embodiments, the secondary liquid 240 can be at least partially miscible with the primary lubricating oil 230. In some embodiments, the secondary liquid 240 can be fully miscible with the primary lubricating oil 230. The secondary liquid 240 can improve the wettability of the primary lubricating oil 230 on substrates such as, but not limited to EVA, PVC, PET, polyurethane, polyethylene, polypropylene, and/or silicone. In some embodiments, the secondary liquid 240 can adjust the viscosity of the primary lubricating oil 230.

In some embodiments, the secondary liquid 240 can make up at least about 1 wt %, at least about 2 wt %, at least about 3 wt %, at least about 4 wt %, at least about 5 wt %, at least about 6 wt %, at least about 7 wt %, at least about 8 wt %, at least about 9 wt %, at least about 10 wt %, at least about 15 wt %, at least about 20 wt %, at least about 25 wt %, at least about 30 wt %, at least about 35 wt %, at least about 40 wt %, or at least about 45 wt % of the lubricating liquid 220. In some embodiments, the secondary liquid 240 can make up no more than about 50 wt %, no more than about 45 wt %, no more than about 40 wt %, no more than about 35 wt %, no more than about 30 wt %, no more than about 25 wt %, no more than about 20 wt %, no more than about 15 wt %, no more than about 10 wt %, no more than about 9 wt %, no more than about 8 wt %, no more than about 7 wt %, no more than about 6 wt %, no more than about 5 wt %, no more than about 4 wt %, no more than about 3 wt %, or no more than about 2 wt % of the lubricating liquid 220. Combinations of the above-referenced weight percentages are also possible (e.g., at least about 1 wt % and no more than about 50 wt % or at least about 10 wt % and no more than about 30 wt %), inclusive of all values and ranges therebetween. In some embodiments, the secondary liquid 240 can make up about 1 wt %, about 2 wt %, about 3 wt %, about 4 wt %, about 5 wt %, about 6 wt %, about 7 wt %, about 8 wt %, about 9 wt %, about 10 wt %, about 15 wt %, about 20 wt %, about 25 wt %, about 30 wt %, about 35 wt %, about 40 wt %, about 45 wt %, or about 50 wt % of the lubricating liquid 220.

In some embodiments, the secondary liquid 240 can include a natural liquid and/or a synthetic liquid. In some embodiments, the secondary liquid 240 can include a fatty acid, a fatty acid ester, vitamin E, vitamin D, cholesterol, a polyolefin, a hydrogenated polyolefin, an ester, a silicone, a fluorocarbon, or any combination thereof.

The antioxidant 222 and the polymer 224 are integrated into the primary lubricating oil 230 and the secondary liquid 240. In some embodiments, the antioxidant 222, the polymer 224, and/or the deodorant 226 can be co-dissolved in the mixture of the primary lubricating oil 230 and the secondary liquid 240. In some embodiments, the antioxidant 222 and the polymer can be co-suspended in the mixture of the primary lubricating oil 230 and the secondary liquid 240.

In some embodiments, the antioxidant 222 can make up at least about 0.01 wt %, at least about 0.02 wt %, at least about 0.03 wt %, at least about 0.04 wt %, at least about 0.05 wt %, at least about 0.06 wt %, at least about 0.07 wt %, at least about 0.08 wt %, at least about 0.09 wt %, at least about 0.1 wt %, at least about 0.2 wt %, at least about 0.3 wt %, at least about 0.4 wt %, at least about 0.5 wt %, at least about 0.6 wt %, at least about 0.7 wt %, at least about 0.8 wt %, at least about 0.9 wt %, at least about 1 wt %, at least about 1.1 wt %, at least about 1.2 wt %, at least about 1.3 wt %, at least about 1.4 wt %, at least about 1.5 wt %, at least about 1.6 wt %, at least about 1.7 wt %, at least about 1.8 wt %, or at least about 1.9 wt % of the lubricating liquid 220. In some embodiments, the antioxidant 222 can make up no more than about 2 wt %, no more than about 1.9 wt %, no more than about 1.8 wt %, no more than about 1.7 wt %, no more than about 1.6 wt %, no more than about 1.5 wt %, no more than about 1.4 wt %, no more than about 1.3 wt %, no more than about 1.2 wt %, no more than about 1.1 wt %, no more than about 1 wt %, no more than about 0.9 wt %, no more than about 0.8 wt %, no more than about 0.7 wt %, no more than about 0.6 wt %, no more than about 0.5 wt %, no more than about 0.4 wt %, no more than about 0.3 wt %, no more than about 0.2 wt %, no more than about 0.1 wt %, no more than about 0.09 wt %, no more than about 0.08 wt %, no more than about 0.07 wt %, no more than about 0.06 wt %, no more than about 0.05 wt %, no more than about 0.04 wt %, no more than about 0.03 wt %, or no more than about 0.02 wt % of the lubricating liquid 220. Combinations of the above-referenced percentages are also possible (e.g., at least about 0.01 wt % and no more than about 2 wt % or at least about 0.1 wt % and no more than about 1 wt %), inclusive of all values and ranges therebetween. In some embodiments, the antioxidant 222 can make up about 0.01 wt %, about 0.02 wt %, about 0.03 wt %, about 0.04 wt %, about 0.05 wt %, about 0.06 wt %, about 0.07 wt %, about 0.08 wt %, about 0.09 wt %, about 0.1 wt %, about 0.2 wt %, about 0.3 wt %, about 0.4 wt %, about 0.5 wt %, about 0.6 wt %, about 0.7 wt %, about 0.8 wt %, about 0.9 wt %, about 1 wt %, about 1.1 wt %, about 1.2 wt %, about 1.3 wt %, about 1.4 wt %, about 1.5 wt %, about 1.6 wt %, about 1.7 wt %, about 1.8 wt %, about 1.9 wt %, or about 2 wt % of the lubricating liquid 220.

In some embodiments, the antioxidant 222 can include a natural compound or derivative. In some embodiments, the antioxidant 222 can include a synthetic compound or derivative. In some embodiments, the antioxidant 222 can include butylated hydroxytoluene (BHT), butylated hydroxyanisole (BHA), tocopherol, ascorbyl palmitate, propyl gallate, ascorbic acid, a phyosterol, oryzanol, citric acid, sesamolin, squalene, tocotrienols, oryzanol, lecithin, or any combination thereof.

The polymer 224 is included in the lubricating liquid 220 and can improve the ability of the lubricating liquid 220 to remain on the target surface during use and between exposures to contact product. In some embodiments, the polymer 224 can make up at least about 0.01 wt %, at least about 0.02 wt %, at least about 0.03 wt %, at least about 0.04 wt %, at least about 0.05 wt %, at least about 0.06 wt %, at least about 0.07 wt %, at least about 0.08 wt %, at least about 0.09 wt %, at least about 0.1 wt %, at least about 0.2 wt %, at least about 0.3 wt %, at least about 0.4 wt %, at least about 0.5 wt %, at least about 0.6 wt %, at least about 0.7 wt %, at least about 0.8 wt %, at least about 0.9 wt %, at least about 1 wt %, at least about 2 wt %, at least about 3 wt %, at least about 4 wt %, at least about 5 wt %, at least about 6 wt %, at least about 7 wt %, at least about 8 wt %, or at least about 9 wt % of the lubricating liquid 220. In some embodiments, the polymer 224 can make up no more than about 10 wt %, no more than about 9 wt %, no more than about 8 wt %, no more than about 7 wt %, no more than about 6 wt %, no more than about 5 wt %, no more than about 4 wt %, no more than about 3 wt %, no more than about 2 wt %, no more than about 1 wt %, no more than about 0.9 wt %, no more than about 0.8 wt %, no more than about 0.7 wt %, no more than about 0.6 wt %, no more than about 0.5 wt %, no more than about 0.4 wt %, no more than about 0.3 wt %, no more than about 0.2 wt %, no more than about 0.1 wt %, no more than about 0.09 wt %, no more than about 0.08 wt %, no more than about 0.07 wt %, no more than about 0.06 wt %, no more than about 0.05 wt %, no more than about 0.04 wt %, no more than about 0.03 wt %, or no more than about 0.02 wt % of the lubricating liquid 220. Combinations of the above-referenced weight percentages are also possible (e.g., at least about 0.01 wt % and no more than about 10 wt % or at least about 0.5 wt % and no more than about 5 wt %), inclusive of all values and ranges therebetween. In some embodiments, the polymer 224 can make up about 0.01 wt %, about 0.02 wt %, about 0.03 wt %, about 0.04 wt %, about 0.05 wt %, about 0.06 wt %, about 0.07 wt %, about 0.08 wt %, about 0.09 wt %, about 0.1 wt %, about 0.2 wt %, about 0.3 wt %, about 0.4 wt %, about 0.5 wt %, about 0.6 wt %, about 0.7 wt %, about 0.8 wt %, about 0.9 wt %, about 1 wt %, about 2 wt %, about 3 wt %, about 4 wt %, about 5 wt %, about 6 wt %, about 7 wt %, about 8 wt %, about 9 wt %, or about 10 wt % of the lubricating liquid 220.

In some embodiments, the polymer 224 can include a natural polymer. In some embodiments, the polymer 224 can include a synthetic polymer. In some embodiments, the polymer 224 can include cellulose, a derivative of cellulose, wax, alginate, gelatin, polypeptide, a homopolymer, a copolymer with acrylic acid, an acrylic ester, an allyl acid, an allyl ester, a carboxylic acid, hydroxyl, vinyl alcohol, ethylene glycol, propylene glycol, and/or a compound including ester moieties.

In some embodiments, the polymer 224 can increase the viscosity of the lubricating liquid 220 by at least about 10%, at least about 15%, at least about 20%, at least about 25%, at least about 30%, at least about 35%, at least about 40%, at least about 45%, at least about 50%, at least about 55%, or at least about 60% as compared to the lubricating liquid 220 without the polymer 224, inclusive of all values and ranges therebetween. In some embodiments, the polymer 224 can dissolve in the primary lubricating oil 230 and/or the secondary liquid 240 and form physical crosslinking via hydrogen bonding. In some embodiments, the polymer 224 can form particles in the lubricating liquid 220. In some embodiments, the polymer 224 can dissolve in the primary lubricating oil 230 and/or the secondary liquid 240 and form a physical gel via hydrogen bonding. The crosslinking can be demonstrated via a crossover of storage modulus and loss modulus, as measured by a rheometer.

In some embodiments, the lubricating liquid 220 can include a deodorant 226. In some embodiments, the deodorant 226 can include chlorophyllin copper, copper citrate, copper gluconate, N-soya-N-ethyl morpholinium ethosulfate (Forestall™), vegetable protein extracts (including but not limited to sodium caproyl/lauroyl lactylate, sodium decanoyl/dodecanoyl lactylate), triethyl citrate, essential oils (including but not limited to pine oil, sage oil eucalyptus oil, Melaleuca armillaris, peppermint oil, and/or lavender oil), or any combination thereof.

In some embodiments, the deodorant 226 can make up at least about 0.01 wt %, at least about 0.02 wt %, at least about 0.03 wt %, at least about 0.04 wt %, at least about 0.05 wt %, at least about 0.06 wt %, at least about 0.07 wt %, at least about 0.08 wt %, at least about 0.09 wt %, at least about 0.1 wt %, at least about 0.2 wt %, at least about 0.3 wt %, at least about 0.4 wt %, at least about 0.5 wt %, at least about 0.6 wt %, at least about 0.7 wt %, at least about 0.8 wt %, at least about 0.9 wt %, at least about 1 wt %, at least about 1.1 wt %, at least about 1.2 wt %, at least about 1.3 wt %, at least about 1.4 wt %, at least about 1.5 wt %, at least about 1.6 wt %, at least about 1.7 wt %, at least about 1.8 wt %, or at least about 1.9 wt % of the lubricating liquid 220. In some embodiments, the deodorant 226 can make up no more than about 2 wt %, no more than about 1.9 wt %, no more than about 1.8 wt %, no more than about 1.7 wt %, no more than about 1.6 wt %, no more than about 1.5 wt %, no more than about 1.4 wt %, no more than about 1.3 wt %, no more than about 1.2 wt %, no more than about 1.1 wt %, no more than about 1 wt %, no more than about 0.9 wt %, no more than about 0.8 wt %, no more than about 0.7 wt %, no more than about 0.6 wt %, no more than about 0.5 wt %, no more than about 0.4 wt %, no more than about 0.3 wt %, no more than about 0.2 wt %, no more than about 0.1 wt %, no more than about 0.09 wt %, no more than about 0.08 wt %, no more than about 0.07 wt %, no more than about 0.06 wt %, no more than about 0.05 wt %, no more than about 0.04 wt %, no more than about 0.03 wt %, or no more than about 0.02 wt % of the lubricating liquid 220. Combinations of the above-referenced percentages are also possible (e.g., at least about 0.01 wt % and no more than about 2 wt % or at least about 0.1 wt % and no more than about 1 wt %), inclusive of all values and ranges therebetween. In some embodiments, the deodorant 226 can make up about 0.01 wt %, about 0.02 wt %, about 0.03 wt %, about 0.04 wt %, about 0.05 wt %, about 0.06 wt %, about 0.07 wt %, about 0.08 wt %, about 0.09 wt %, about 0.1 wt %, about 0.2 wt %, about 0.3 wt %, about 0.4 wt %, about 0.5 wt %, about 0.6 wt %, about 0.7 wt %, about 0.8 wt %, about 0.9 wt %, about 1 wt %, about 1.1 wt %, about 1.2 wt %, about 1.3 wt %, about 1.4 wt %, about 1.5 wt %, about 1.6 wt %, about 1.7 wt %, about 1.8 wt %, about 1.9 wt %, or about 2 wt % of the lubricating liquid 220.

In some embodiments, the lubricating liquid 220 can execute shear thinning behavior. In some embodiments, the lubricating liquid 220 can have a shear thinning index (calculated as the apparent viscosity at a speed of 2 rpm divided by the apparent viscosity at a speed of 20 rpm) of about 1.1, about 1.2, about 1.3, about 1.4, about 1.5, about 1.6, about 1.7, about 1.8, about 1.9, about 2, about 2.5, about 3, about 3.5, about 4, about 4.5, about 5, about 5.5, about 6, about 6.5, about 7, about 7.5, about 8, about 8.5, about 9, about 9.5, or about 10, inclusive of all values and ranges therebetween. In some embodiments, the lubricating liquid can have a wettability on EVA film with a receding contact angle in air of less than about 15°, less than about 14°, less than about 13°, less than about 12°, less than about 11°, less than about 10°, less than about 9°, less than about 8°, less than about 7°, less than about 6°, or less than about 5°, inclusive of all values and ranges therebetween. In some embodiments, the lubricating liquid 220 can have a stability on EVA film in the presence of a viscous product.

In some embodiments, the lubricating liquid 220 can have a yield stress at 20° C. of at least about 0.01 Pa, at least about 0.02 Pa, at least about 0.03 Pa, at least about 0.04 Pa, at least about 0.05 Pa, at least about 0.06 Pa, at least about 0.07 Pa, at least about 0.08 Pa, at least about 0.09 Pa, at least about 0.1 Pa, at least about 0.15 Pa, at least about 0.2 Pa, at least about 0.25 Pa, at least about 0.3 Pa, at least about 0.35 Pa, at least about 0.4 Pa, at least about 0.45 Pa, at least about 0.5 Pa, at least about 0.55 Pa, at least about 0.6 Pa, at least about 0.65 Pa, at least about 0.7 Pa, at least about 0.75 Pa, at least about 0.8 Pa, at least about 0.85 Pa, at least about 0.9 Pa, or at least about 0.95 Pa. In some embodiments, the lubricating liquid 220 can have a yield stress at 20° C. of no more than about 1 Pa, no more than about 0.95 Pa, no more than about 0.9 Pa, no more than about 0.85 Pa, no more than about 0.8 Pa, no more than about 0.75 Pa, no more than about 0.7 Pa, no more than about 0.65 Pa, no more than about 0.6 Pa, no more than about 0.55 Pa, no more than about 0.5 Pa, no more than about 0.45 Pa, no more than about 0.4 Pa, no more than about 0.35 Pa, no more than about 0.3 Pa, no more than about 0.25 Pa, no more than about 0.2 Pa, no more than about 0.15 Pa, no more than about 0.10 Pa, no more than about 0.09 Pa, no more than about 0.08 Pa, no more than about 0.07 Pa, no more than about 0.06 Pa, no more than about 0.05 Pa, no more than about 0.04 Pa, no more than about 0.03 Pa, or no more than about 0.02 Pa. Combinations of the above-referenced yield stress values are also possible (e.g., at least about 0.1 Pa and no more than about 1 Pa or at least about 0.3 Pa and no more than about 0.7 Pa), inclusive of all values and ranges therebetween. In some embodiments, the lubricating liquid 220 can have a yield stress at 20° C. of about 0.01 Pa, about 0.02 Pa, about 0.03 Pa, about 0.04 Pa, about 0.05 Pa, about 0.06 Pa, about 0.07 Pa, about 0.08 Pa, about 0.09 Pa, about 0.1 Pa, about 0.15 Pa, about 0.2 Pa, about 0.25 Pa, about 0.3 Pa, about 0.35 Pa, about 0.4 Pa, about 0.45 Pa, about 0.5 Pa, about 0.55 Pa, about 0.6 Pa, about 0.65 Pa, about 0.7 Pa, about 0.75 Pa, about 0.8 Pa, about 0.85 Pa, about 0.9 Pa, about 0.95 Pa, or about 1 Pa.

In some embodiments, the lubricating liquid 220 can have a viscosity at 20° C. when measured at any shear rate between 100/s and 1000/s of at least about 0.02 Pa·s, at least about 0.03 Pa·s, at least about 0.04 Pa·s, at least about 0.05 Pa·s, at least about 0.06 Pa·s, at least about 0.07 Pa·s, at least about 0.08 Pa·s, at least about 0.09 Pa·s, at least about 0.1 Pa·s, at least about 0.11 Pa·s, at least about 0.12 Pa·s, at least about 0.13 Pa·s, at least about 0.14 Pa·s, at least about 0.15 Pa·s, at least about 0.16 Pa·s, at least about 0.17 Pa·s, at least about 0.18 Pa·s, or at least about 0.19 Pa·s. In some embodiments, the lubricating liquid 220 can have a viscosity at 20° C. when measured at a shear rate of 1000/s of no more than about 0.2 Pa·s, no more than about 0.19 Pa·s, no more than about 0.18 Pa·s, no more than about 0.17 Pa·s, no more than about 0.16 Pa·s, no more than about 0.15 Pa·s, no more than about 0.14 Pa·s, no more than about 0.13 Pa·s, no more than about 0.12 Pa·s, no more than about 0.11 Pa·s, no more than about 0.1 Pa·s, no more than about 0.09 Pa·s, no more than about 0.08 Pa·s, no more than about 0.07 Pa·s, no more than about 0.06 Pa·s, no more than about 0.05 Pa·s, no more than about 0.04 Pa·s, or no more than about 0.03 Pa·s. Combinations of the above-referenced viscosity values are also possible (e.g., at least about 0.02 Pa·s and no more than about 0.2 Pa·s or at least about 0.05 Pa·s and no more than about 0.15 Pa·s), inclusive of all values and ranges therebetween. In some embodiments, the lubricating liquid 220 can have a viscosity at 20° C. when measured at any shear rate between 100/s and 1000/s of about 0.02 Pa·s, about 0.03 Pa·s, about 0.04 Pa·s, about 0.05 Pa·s, about 0.06 Pa·s, about 0.07 Pa: s, about 0.08 Pa·s, about 0.09 Pa·s, about 0.1 Pa·s, about 0.11 Pa·s, about 0.12 Pa·s, about 0.13 Pa·s, about 0.14 Pa·s, about 0.15 Pa·s, about 0.16 Pa·s, about 0.17 Pa·s, about 0.18 Pa·s, about 0.19 Pa·s, or about 0.2 Pa·s.

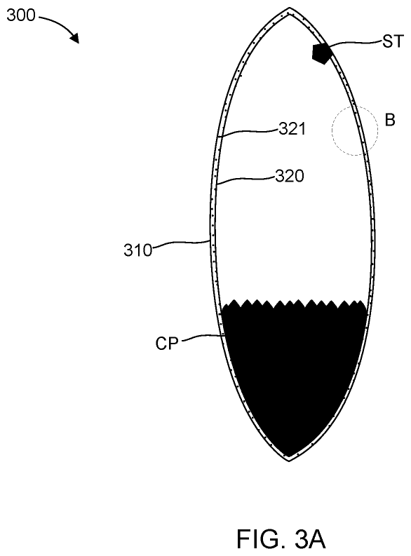

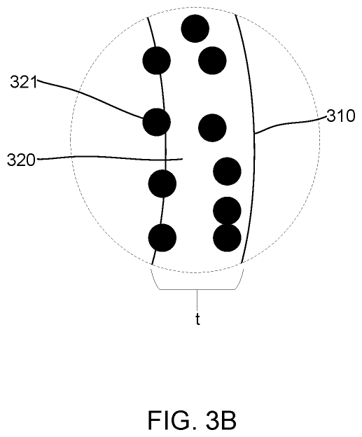

FIGS. 3A-3B are illustrations of an ostomy pouch 300, according to an embodiment. As shown, the ostomy pouch 300 includes a target surface 310 with a lubricating liquid 320 disposed thereon. Particles 321 are disposed in the lubricating liquid 320. As shown, a contact product CP is held inside the ostomy pouch 300 and a stoma ST protrudes into the ostomy pouch 300. FIG. 3A shows a cross-sectional view of the ostomy pouch 300, while FIG. 3B shows a close-up view of a layer of the lubricating liquid 320, as indicated by the circle B, as shown in FIG. 3A.

As shown, the lubricating liquid 320 is coated on the inner surface of the target surface 310 with a thickness t. In some embodiments, the lubricating liquid 320, in the absence of a contacting phase, exhibits a maximum shear stress at the interface between the lubricating liquid 320 and the target surface. The maximum shear stress can be equal to the coating weight per unit area, as described by the following equation.

$${τ}_{max} = ρ * g * t$$

Where τₘₐₓ is maximum shear stress;
ρ is density (kg/m²);
g is the acceleration due to gravity (9.8 m/s²); and
t is the thickness of the coating layer of the lubricating liquid 320.

As long as the yield stress of the lubricating liquid 320 is greater than the τₘₐₓ, then the lubricating liquid 320 does not flow (in the absence of a product (e.g., the contact product CP)). That is, the coating layer of the lubricating liquid 320 is engineered such that the yield stress of the lubricating liquid 320 is greater than or equal to τₘₐₓ. For a 10 μm thickness lubricating liquid 320, the yield stress of the lubricating liquid 320 should be at least 0.1 Pa. For a 30 μm thickness lubricating liquid 230, the yield stress of the lubricating liquid 320 should be at least 0.3 Pa. However, increasing the yield stress of the lubricating liquid 320 can also increase the viscosity of the lubricating liquid. It is important to achieve a viscosity low enough to allow sliding of the contact product CP from the lubricating liquid 320 at a relatively high speed (i.e., to prevent pancaking near the stoma ST). The viscosity of the lubricating liquid 320 should be sufficiently low at a relevant shear rate (i.e., the shear rate experienced by the lubricating liquid 320 as the contact product CP slides over the lubricating liquid 320). A desired shear rate can be determined by considering a desired sliding speed of the contact product CP. In the case of an ostomy pouch, sliding speeds of at least about 1 cm/s are preferable. The relevant shear rate can be approximated as a sliding speed divided by the thickness t. For a 10 μm coating, with an interface speed of 1 cm/s, the shear rate would be 1000/s. For a Type 3 or a Type 4 stool, the stool typically travels down and through the ostomy pouch with a stool thickness of about 1 cm. The stool would therefore impart a stress on the interface between the stool and the lubricating liquid 320 of ρ*g*tₛₜₒₒₗ, which would equal about 100 Pa. The coating viscosity that achieves such a sliding speed is defined by Newton's law of viscosity, as defined below.

$$μ = τ / \dot{γ}$$

Where μ is viscosity (Pa·s);
τ is shear stress (Pa);
\dot{γ} is shear rate (s⁻¹).

For a 1 cm thick stool sliding on a 10 μm coating, the coating viscosity that would provide a sliding speed of at least 1 cm/s would be 0.1 Pa·s or less, where the viscosity is measured at the relevant shear rate (\dot{γ}=1,000/s). For example, the viscosity of some lubricating liquids shear thin to a minimum value at shear rates of greater than 10/s, and the minimum viscosity of the lubricant solution can have a viscosity rate of 0.023-0.043 Pa·s at these high shear rates. Further the stool drop time of Type 3 stool can have a range of 20 seconds to 80 seconds for pouches lubricated with various lubricating solutions, which corresponds to a sliding speed of 0.2-0.7 cm/s for a standard pouch size. An example fully lubricated pouch can have a lubricant thickness in the range of 10 μm to about 40 μm (corresponding to a volume or lubricating liquid of 0.5-2 mL). For this range of sliding speed and lubricant thickness, the shear rate imparted on the lubricant due to the sliding of the stool is greater than 40/s. Therefore, the viscosity of the lubricant is expected to be at a minimum while the stool slides down the pouch. Similarly, a 100 μm coating can achieve a 1 cm/s slide rate if the viscosity is 1 Pa·s. However, such a yield stress would render importance of a higher yield stress of 1 Pa to be stabilized. In addition to the rheology characteristics, the lubricating liquid 320 can be engineered to achieve desirable wettability, and to be sufficiently immiscible with the stool.

In some embodiments, the thickness t can be at least about 1 μm, at least about 2 μm, at least about 3 μm, at least about 4 μm, at least about 5 μm, at least about 6 μm, at least about 7 μm, at least about 8 μm, at least about 9 μm, at least about 10 μm, at least about 20 μm, at least about 30 μm, at least about 40 μm, at least about 50 μm, at least about 60 μm, at least about 70 μm, at least about 80 μm, at least about 90 μm, at least about 100 μm, at least about 200 μm, at least about 300 μm, at least about 400 μm, at least about 500 μm, at least about 600 μm, at least about 700 μm, at least about 800 μm, at least about 900 μm, at least about 1 mm, at least about 2 mm, at least about 3 mm, at least about 4 mm, at least about 5 mm, at least about 6 mm, at least about 7 mm, at least about 8 mm, or at least about 9 mm. In some embodiments, the thickness t can be no more than about 1 cm, no more than about 9 mm, no more than about 8 mm, no more than about 7 mm, no more than about 6 mm, no more than about 5 mm, no more than about 4 mm, no more than about 3 mm, no more than about 2 mm, no more than about 1 mm, no more than about 900 μm, no more than about 800 μm, no more than about 700 μm, no more than about 600 μm, no more than about 500 μm, no more than about 400 μm, no more than about 300 μm, no more than about 200 μm, no more than about 100 μm, no more than about 90 μm, no more than about 80 μm, no more than about 70 μm, no more than about 60 μm, no more than about 50 μm, no more than about 40 μm, no more than about 30 μm, no more than about 20 μm, no more than about 10 μm, no more than about 9 μm, no more than about 8 μm, no more than about 7 μm, no more than about 6 μm, no more than about 5 μm, no more than about 4 μm, no more than about 3 μm, or no more than about 2 μm. Combinations of the above-referenced thicknesses t are also possible (e.g., at least about 1 μm and no more than about 1 cm or at least about 50 μm and no more than about 5 mm), inclusive of all values and ranges therebetween. In some embodiments, the thickness t can be about 1 μm, about 2 μm, about 3 μm, about 4 μm, about 5 μm, about 6 μm, about 7 μm, about 8 μm, about 9 μm, about 10 μm, about 20 μm, about 30 μm, about 40 μm, about 50 μm, about 60 μm, about 70 μm, about 80 μm, about 90 μm, about 100 μm, about 200 μm, about 300 μm, about 400 μm, about 500 μm, about 600 μm, about 700 μm, about 800 μm, about 900 μm, about 1 mm, about 2 mm, about 3 mm, about 4 mm, about 5 mm, about 6 mm, about 7 mm, about 8 mm, about 9 mm, or about 1 cm.

The particles 321 are an optional feature and are disposed in the lubricating liquid 320. In some embodiments, the particles 321 can be formed at least partially from a polymer (e.g., the polymer 224, as described above with reference to FIG. 2). In some embodiments, the particles 321 can be formed at least partially from an antioxidant (e.g., the antioxidant 222, as described above with reference to FIG. 2).

In some embodiments, the particles 321 can have a particle size of at least about 1 μm, at least about 2 μm, at least about 3 μm, at least about 4 μm, at least about 5 μm, at least about 6 μm, at least about 7 μm, at least about 8 μm, at least about 9 μm, at least about 10 μm, at least about 20 μm, at least about 30 μm, at least about 40 μm, at least about 50 μm, at least about 60 μm, at least about 70 μm, at least about 80 μm, or at least about 90 μm. In some embodiments, the particles 321 can have a particle size of no more than about 100 μm, no more than about 90 μm, no more than about 80 μm, no more than about 70 μm, no more than about 60 μm, no more than about 50 μm, no more than about 40 μm, no more than about 30 μm, no more than about 20 μm, no more than about 10 μm, no more than about 9 μm, no more than about 8 μm, no more than about 7 μm, no more than about 6 μm, no more than about 5 μm, no more than about 4 μm, no more than about 3 μm, or no more than about 2 μm. Combinations of the above-referenced particle sizes are also possible (e.g., at least about 1 μm and no more than about 100 μm or at least about 10 μm and no more than about 80 μm), inclusive of all values and ranges therebetween. In some embodiments, the particles 321 can have a particle size of about 1 μm, about 2 μm, about 3 μm, about 4 μm, about 5 μm, about 6 μm, about 7 μm, about 8 μm, about 9 μm, about 10 μm, about 20 μm, about 30 μm, about 40 μm, about 50 μm, about 60 μm, about 70 μm, about 80 μm, about 90 μm, or about 100 μm.

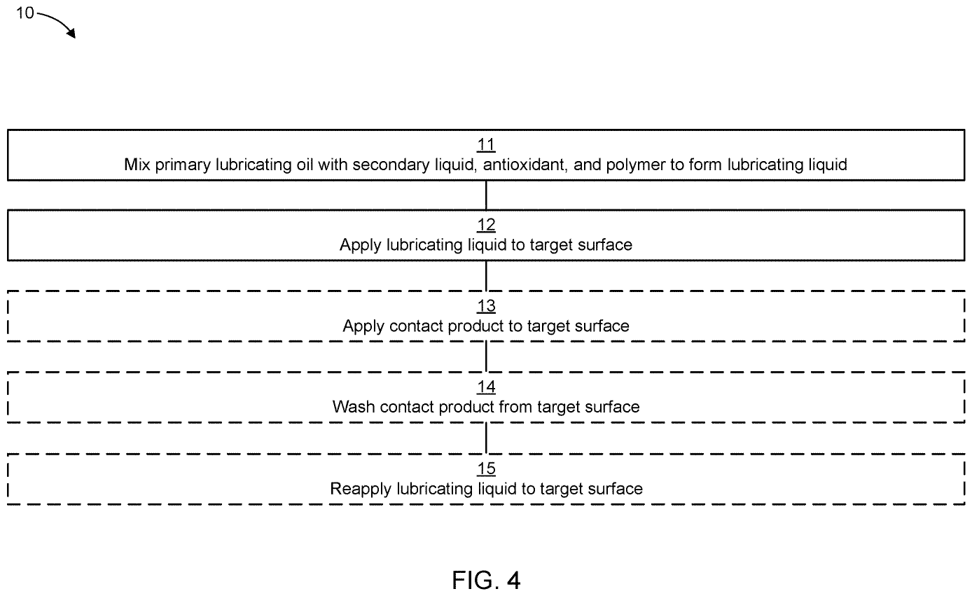

FIG. 4 is a flow diagram of a method 10 of producing a lubricated surface, according to an embodiment. As shown, the method 10 includes mixing a primary lubricating oil with a secondary liquid, an antioxidant, and a polymer to form a lubricating liquid at step 11 and applying the lubricating liquid to a target surface at step 12. The method 10 optionally includes applying a contact product to the contact surface at step 13, washing the contact product from the target surface at step 14, and reapplying the lubricating liquid to the target surface at step 15.

Step 11 includes mixing the primary lubricating oil with the secondary liquid, the antioxidant, and the polymer. In some embodiments, the primary lubricating oil, the secondary liquid, the antioxidant, and the polymer can be the same or substantially similar to the primary lubricating oil 230, the secondary liquid 240, the antioxidant 222, and the polymer 224, as described above with reference to FIG. 2. Thus, certain aspects of the primary lubricating oil, the secondary liquid, the antioxidant, and the polymer are not described in greater detail herein. In some embodiments, the primary lubricating oil can be mixed with the secondary liquid prior to adding the antioxidant and the polymer. In some embodiments, the primary lubricating oil, the secondary liquid, the antioxidant, and the polymer can all be mixed together simultaneously. In some embodiments, the polymer can be dissolved in the mixture that includes the primary lubricating oil and the secondary liquid. In some embodiments, the polymer can be suspended in the mixture that includes the primary lubricating oil and the secondary liquid, such that the polymer forms particles.

Step 12 includes applying the lubricating liquid to the target surface. In some embodiments, the target surface can include an interior of an ostomy bag, a commode chair, a potty training product (e.g., a miniature toddler toilet or a toddler toilet seat), a bedpan, a camper toilet, a waterless toilet, a space toilet, a fecal catheter, a surgical bulb, a stent, a pet waste collection scoop, a pet waste collection bag, and/or a catheter. In some embodiments, the target surface can be coated with the lubricating liquid during manufacture of the article that includes the target surface. In some embodiments, the target surface can be coated with the lubricating liquid prior to packaging, shipment, and/or sale of the article including the target surface. In some embodiments, the lubricating liquid can be applied to the target surface via a sachet system. For example, the lubricating liquid can be placed into a porous bag and applied to the target surface via a smearing motion. In some embodiments, the lubricating liquid can be applied to the target surface via spraying (e.g., an aerosol spray). In some embodiments, the lubricating liquid can be applied to the target surface via a nozzle (e.g., via a squirting motion).

Step 13 is optional and includes applying a contact product to the target surface. In some embodiments, the contact product can have any of the properties of the contact product CP, as described above with reference to FIG. 1. Thus, certain aspects of the contact product are not described in greater detail herein. The lubricating liquid creates a slippery surface, such that the contact product slides down the target surface (i.e., via gravity). In some embodiments, the contact product can slide down the target surface at a speed of at least about 1 mm/s, at least about 2 mm/s, at least about 3 mm/s, at least about 4 mm/s, at least about 5 mm/s, at least about 6 mm/s, at least about 7 mm/s, at least about 8 mm/s, at least about 9 mm/s, at least about 1 cm/s, at least about 2 cm/s, at least about 3 cm/s, at least about 4 cm/s, at least about 5 cm/s, at least about 6 cm/s, at least about 7 cm/s, at least about 8 cm/s, at least about 9 cm/s, or at least about 10 cm/s, inclusive of all values and ranges therebetween.

Step 14 is optional and includes washing the contact product from the target surface. In some embodiments, washing the contact product from the target surface can include spraying a solvent onto the target surface. In some embodiments, the solvent can include a polar solvent. In some embodiments, the solvent can include water, ethanol, isopropyl alcohol, or any combination thereof. In some embodiments, the solvent can be applied to the target surface at an elevated pressure. In some embodiments, the solvent can be pressurized to a pressure of about 1 bar (gauge), about 2 bar, about 3 bar, about 4 bar, about 5 bar, about 6 bar, about 7 bar, about 8 bar, about 9 bar, about 10 bar, about 20 bar, about 30 bar, about 40 bar, about 50 bar, about 60 bar, about 70 bar, about 80 bar, about 90 bar, or about 100 bar, inclusive of all values and ranges therebetween.

Step 15 is optional and includes reapplying the lubricating liquid to the target surface. In some embodiments, the reapplication of the lubricating liquid can be via the same method as the original application of the lubricating liquid. In some embodiments, the reapplication of the lubricating liquid can be via a different method from the original application of the lubricating liquid. In some embodiments, the contact product can be applied to the target surface and the contact product can be washed from the target surface multiple times between reapplications of lubricating liquid. In other words, steps 13 and 14 can be executed multiple times between each application of step 15. Said another way, multiple stool events can occur between reapplication of the lubricating liquid. In some embodiments, the contact product can be applied and washed from the target surface about 1, about 2, about 3, about 4, about 5, about 6, about 7, about 8, about 9, about 10, about 11, about 12, about 13, about 14, about 15, about 16, about 17, about 18, about 19, or about 20 times between applications of the lubricating liquid to the target surface, inclusive of all values and ranges therebetween.

### Examples

Multiple ostomy pouches were lubricated and subjected to six stool drops in a single day. Different lubricants were applied to the pouch surface (generally a total of 4 mL, unless noted otherwise below). Lubricant was not re-applied. Pouches were tested in a vertical orientation with stools of Type 3 and Type 6 on the Bristol scale. The total test time was 600 seconds (10 minutes), with a maximum slide time allowed for slide time and drain time measurements was 300 seconds (5 minutes), each. In other words, tests were terminated at 10 minutes. A controlled squeeze was performed on the tail of each pouch following the draining time to remove excess stool from the tail of the pouch (modeling real use). Stool drop time, stool drain time, and residual stool mass were measured. If two dispensations were performed and stool still failed to evacuate the pouch, the pouch was removed from further testing.

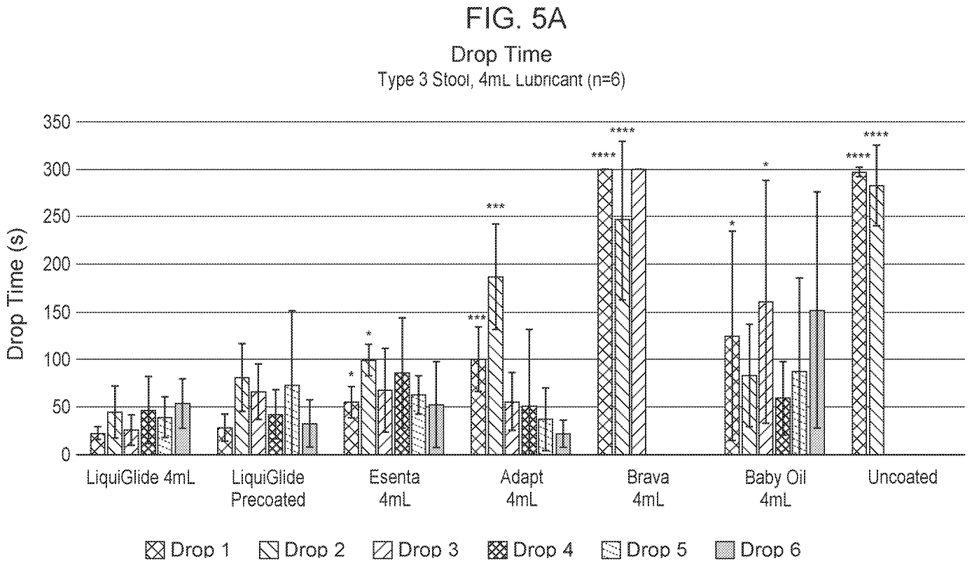

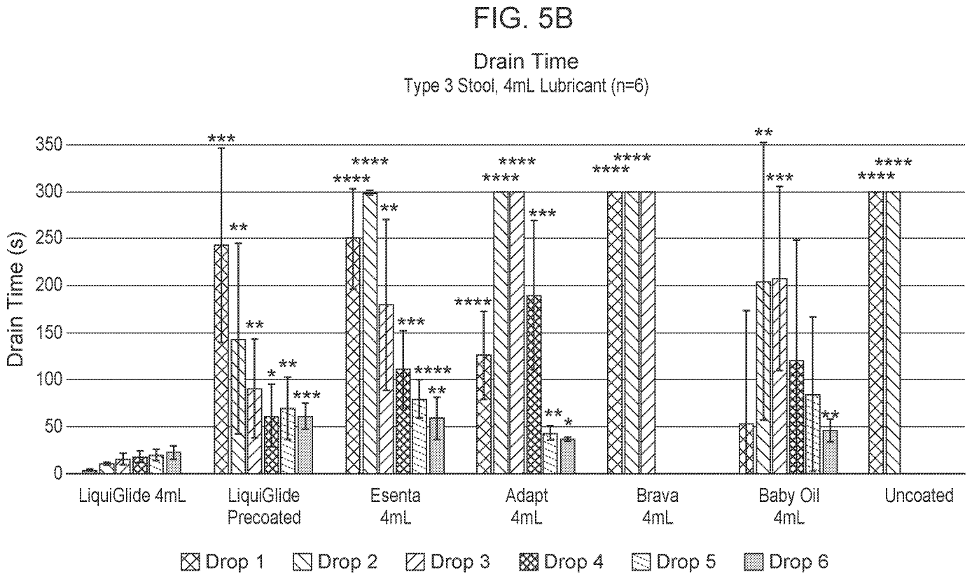

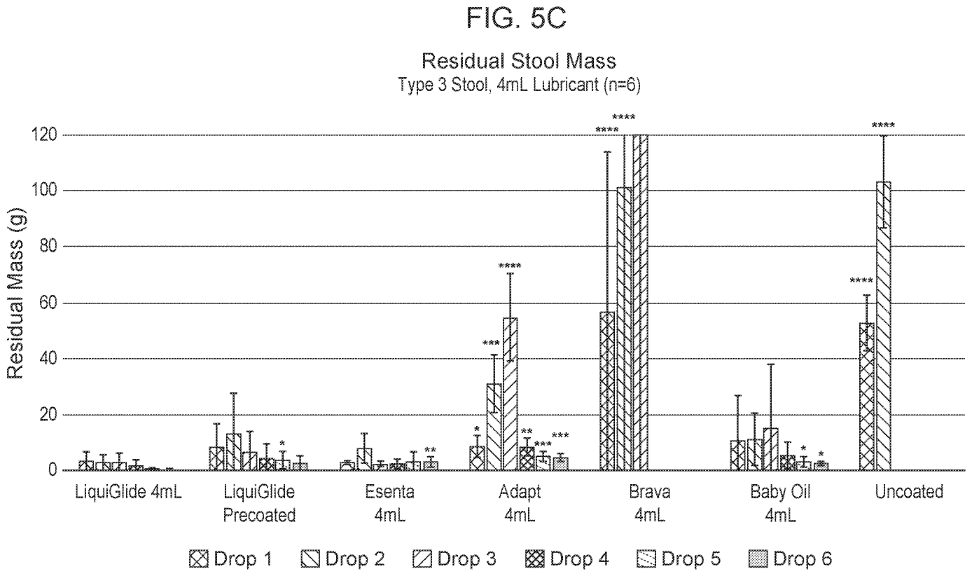

FIG. 5A shows a comparison of drop times of Type 3 stool among five different lubricants, as well as an ostomy pouch with no lubricant. A LiquiGlide lubricant was prepared with about 70 wt % of a vegetable oil as a base, along with about 25 wt % of a fatty acid derivative, with the balance including polymer, antioxidant, and deodorant. The LiquiGlide lubricant was added to the ostomy pouch in an amount of 4 mL prior to running the tests. The LiquiGlide lubricant was compared to commercially available lubricants, specifically Esenta™, Adapt™, and Brava® lubricants, as well as a baby oil coating. An ostomy pouch pre-coated in advance with 0.5 mL of the LiquiGlide ostomy pouch lubricant coating was also tested as a comparison. As shown, the LiquiGlide lubricant consistently maintained stool drop times of less than or close to one minute and had a faster average drop time than each of the other lubricants. The LiquiGlide lubricant was analyzed via a crossover of storage modulus and loss modulus and determined to be about 0.03 Pa.

FIG. 5B shows a comparison of drain times for removal of Type 3 stool from ostomy pouches with the same five lubricants, as well as an ostomy pouch with no lubricant. As shown, the ostomy pouch with 4 mL of LiquiGlide lubricant applied thereto had significantly lower drain times than either of the other pouches. Specifically, the ostomy pouch with 4 mL of LiquiGlide lubricant had drain times that did not exceed 25 seconds through each of the six draining events.

FIG. 5C shows a comparison of residual stool mass after draining of Type 3 stool from ostomy pouches with the same five lubricants, as well as an ostomy pouch with no lubricant. As shown, the ostomy pouch with 4 mL of LiquiGlide lubricant applied thereto had on average the lowest amount of residual stool mass and did not exceed 5 g through each draining event.

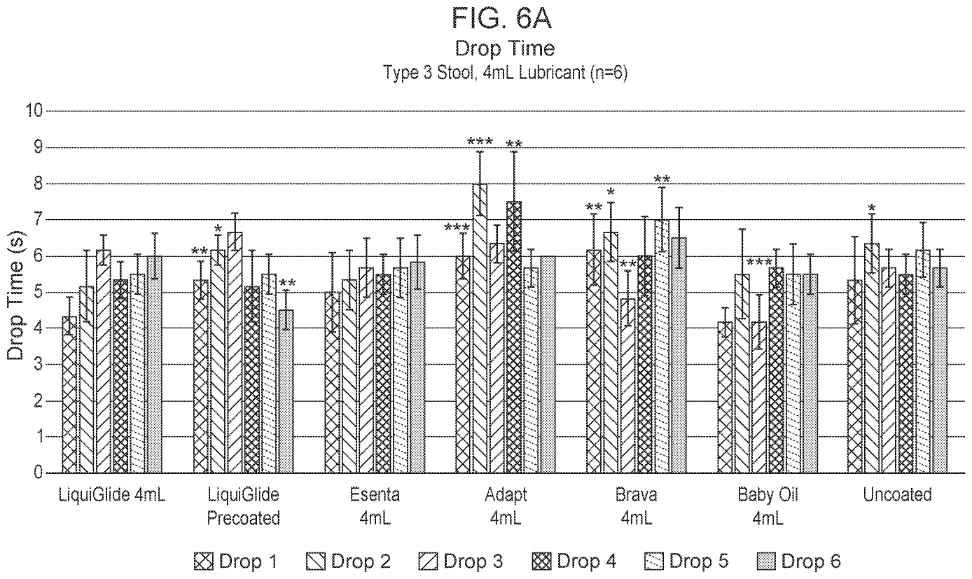

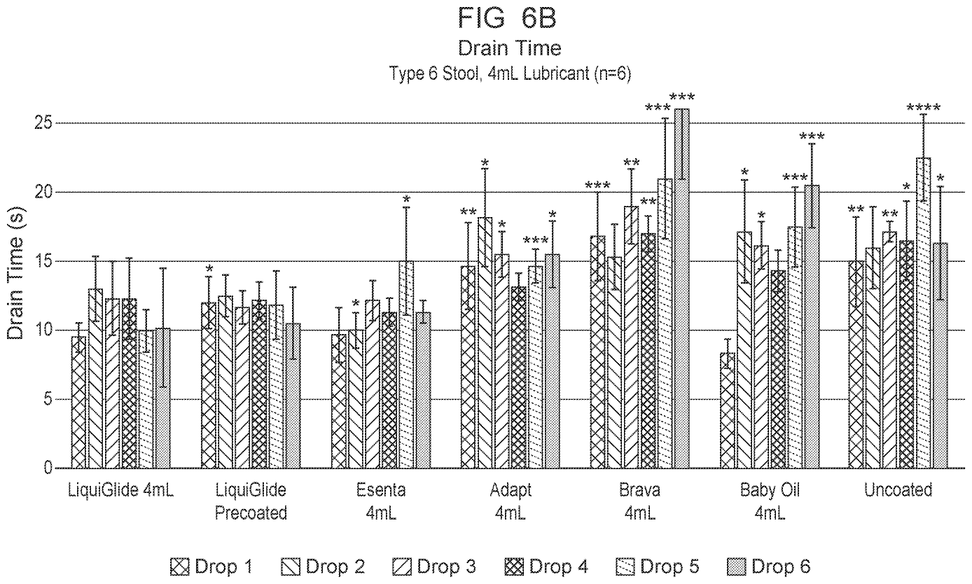

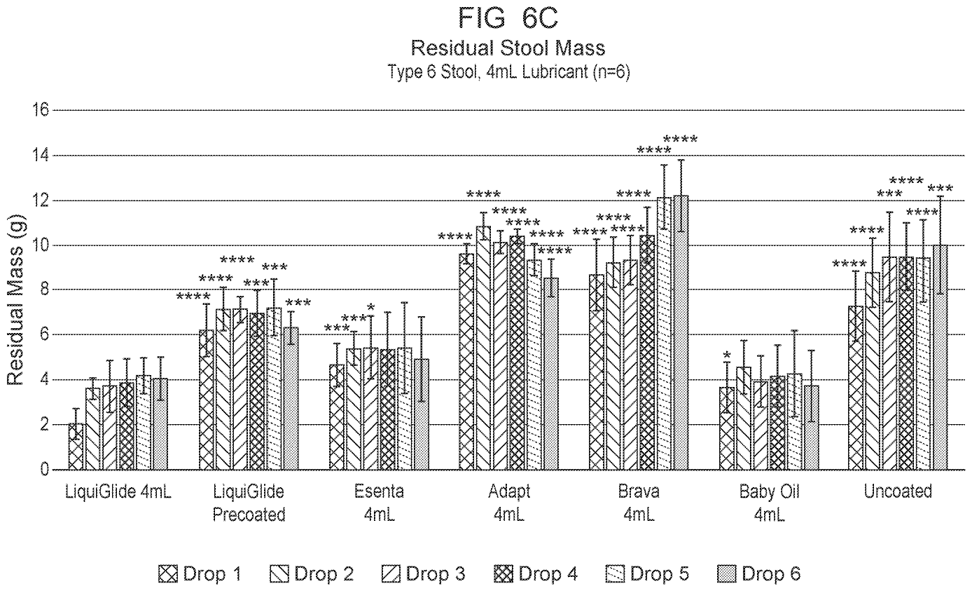

FIG. 6A shows a comparison of drop times of Type 6 stool from ostomy pouches with the same five lubricants, as well as an ostomy pouch with no lubricant. As shown, the drop times across each coated and uncoated pouch are similar, due to the liquidous nature of the stool.

FIG. 6B shows a comparison of drain times for removal of Type 6 stool from ostomy pouches with the same five lubricants, as well as an ostomy pouch with no lubricant. In general, the pouches coated with LiquiGlide lubricant had slightly lower drain times than the other lubricants.

FIG. 6C shows a comparison of residual stool mass after draining of Type 6 stool from ostomy pouches with the same five lubricants, as well as an ostomy pouch with no lubricant. As shown, the pouch coated with 4 mL of LiquiGlide lubricant included on average the lowest amount of residual stool mass, slightly less than the ostomy pouch coated with baby oil.

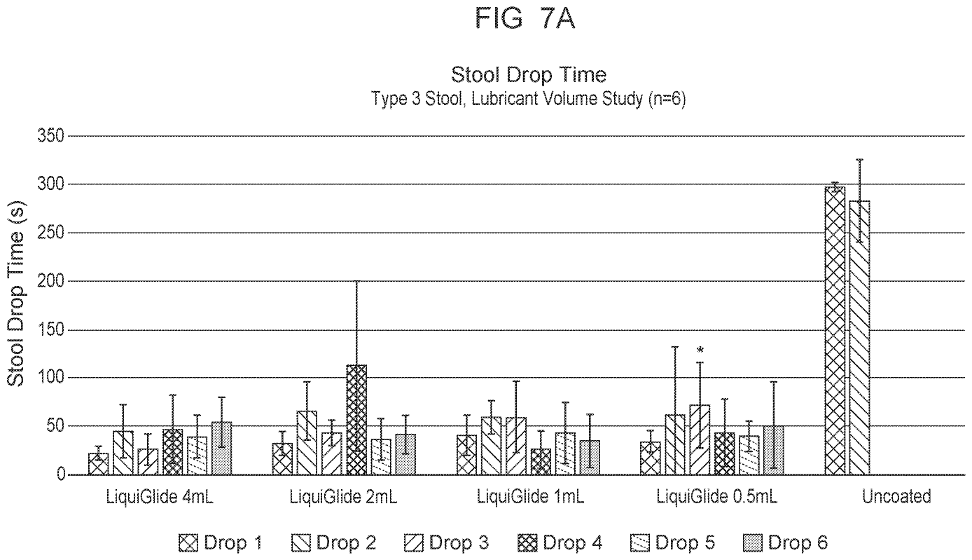

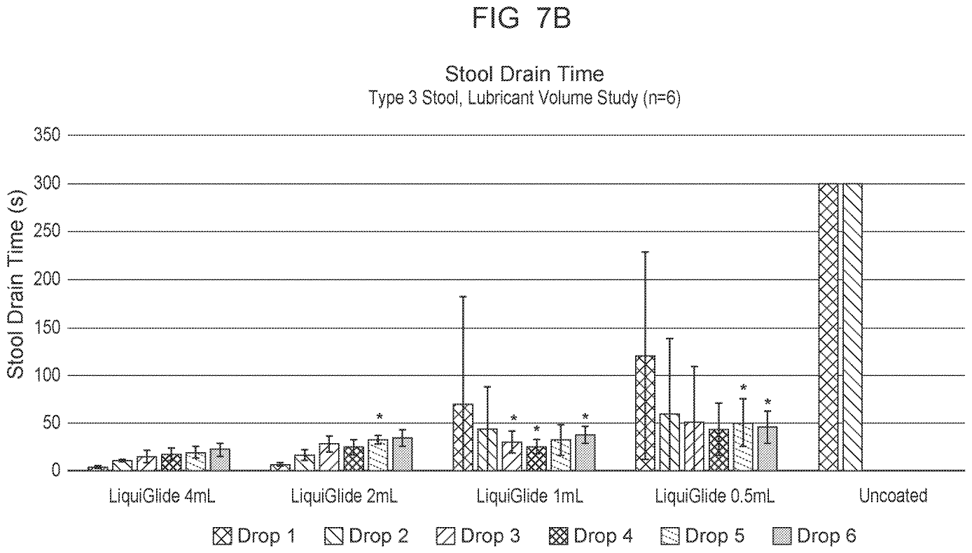

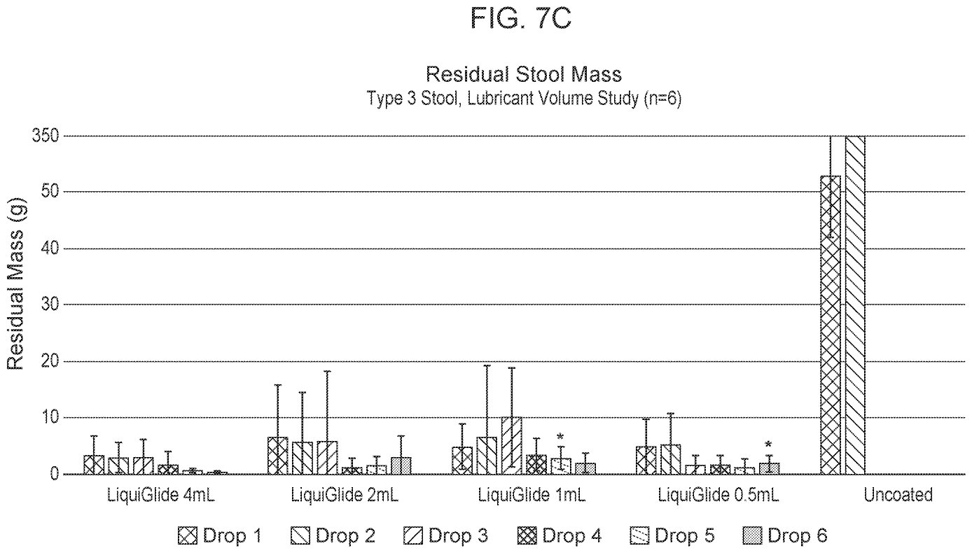

FIG. 7A shows the effect various lubricant doses of LiquiGlide lubricant has on drop times of a Type 3 stool. Ostomy pouches were dosed with 0.5 mL, 1 mL, 2 mL, and 4 mL of LiquiGlide lubricant, and compared to an uncoated ostomy pouch. As shown, stool drop time was fairly consistent regardless of lubricant amount, with a slightly lower average drop time in the pouch coated by 4 mL of LiquiGlide lubricant.

FIG. 7B shows a comparison of drain times of Type 3 stool in ostomy pouches with the same doses of LiquiGlide lubricant. As shown, more lubricant typically leads to faster drain times, with 4 mL of lubricant facilitating the fastest drain times.

FIG. 7C shows a comparison of residual stool mass of Type 3 stool in ostomy pouches with the same doses of LiquiGlide lubricant. As shown, the amount of residual mass is fairly consistent, regardless of the amount of lubricant, with slightly less residual mass on the pouch coated with 4 mL of lubricant.

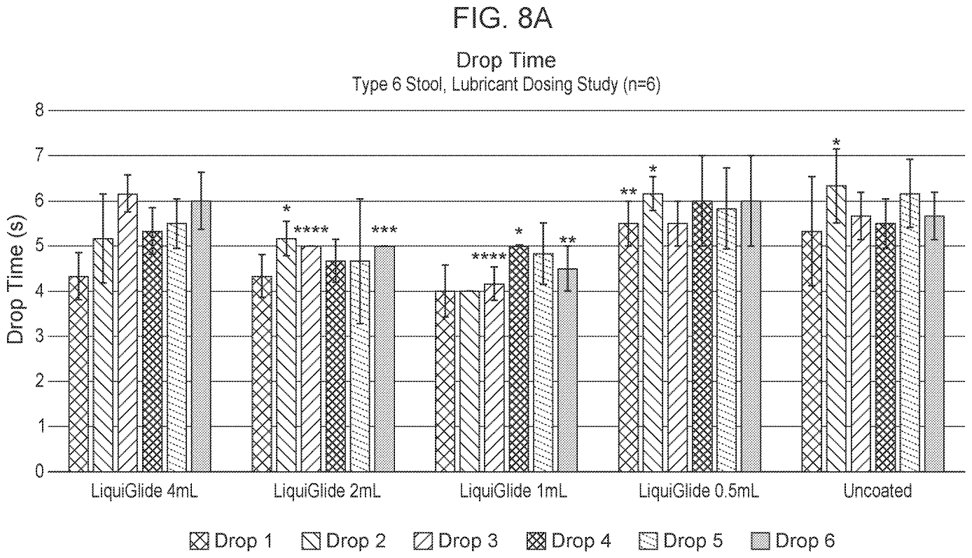

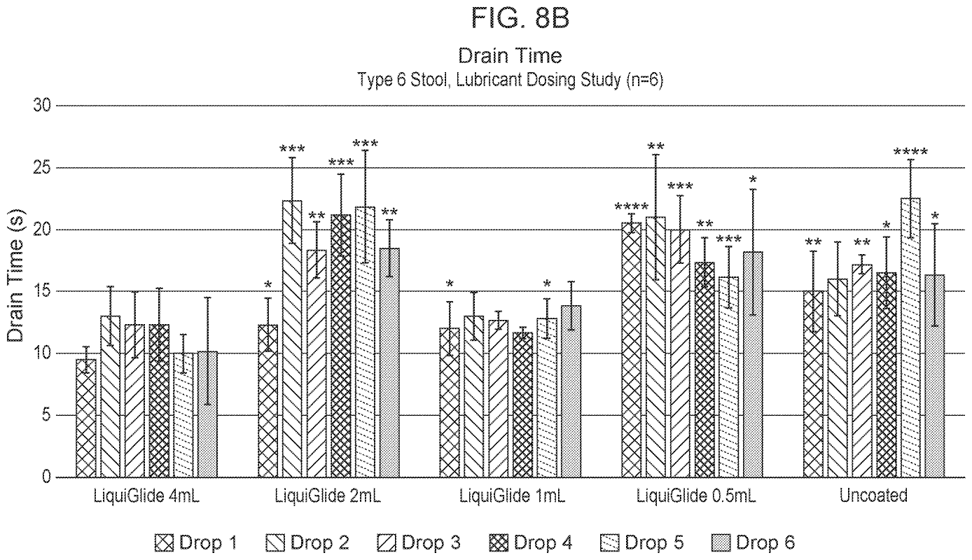

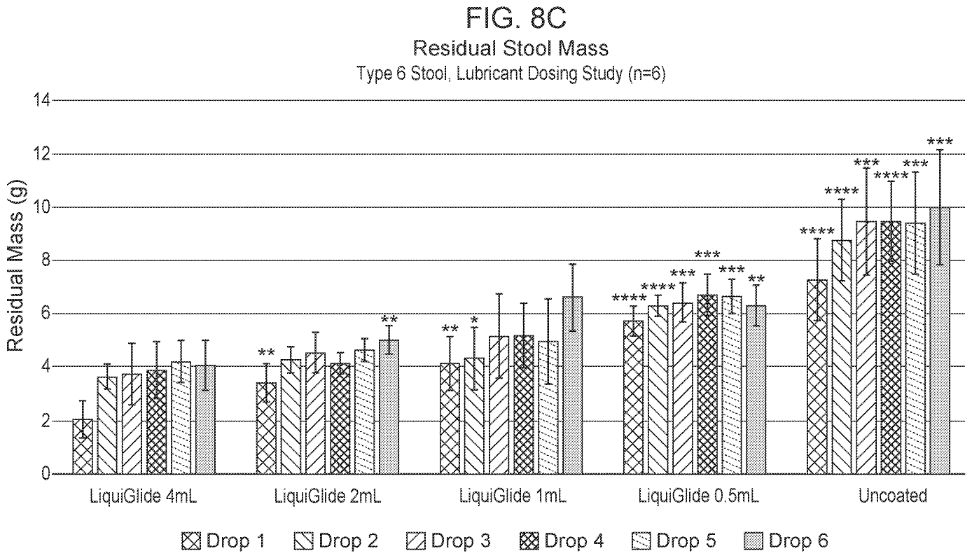

FIG. 8A shows a comparison of the same doses of LiquiGlide lubricant on drop times of a Type 6 stool. As shown, the drop times are relatively unaffected by the amount of lubricant, due to the liquidous nature of the stool.

FIG. 8B shows a comparison of drain times of Type 6 stool in ostomy pouches with the same doses of LiquiGlide lubricant. As shown, the drain times are relatively unaffected by the amount of lubricant, due to the liquidous nature of the stool.

FIG. 8C shows a comparison of residual stool mass of Type 6 stool in ostomy pouches with the same doses of LiquiGlide lubricant. As shown, the ostomy pouch with 4 mL of lubricant had the lowest amount of residual mass, as compared to the other doses.

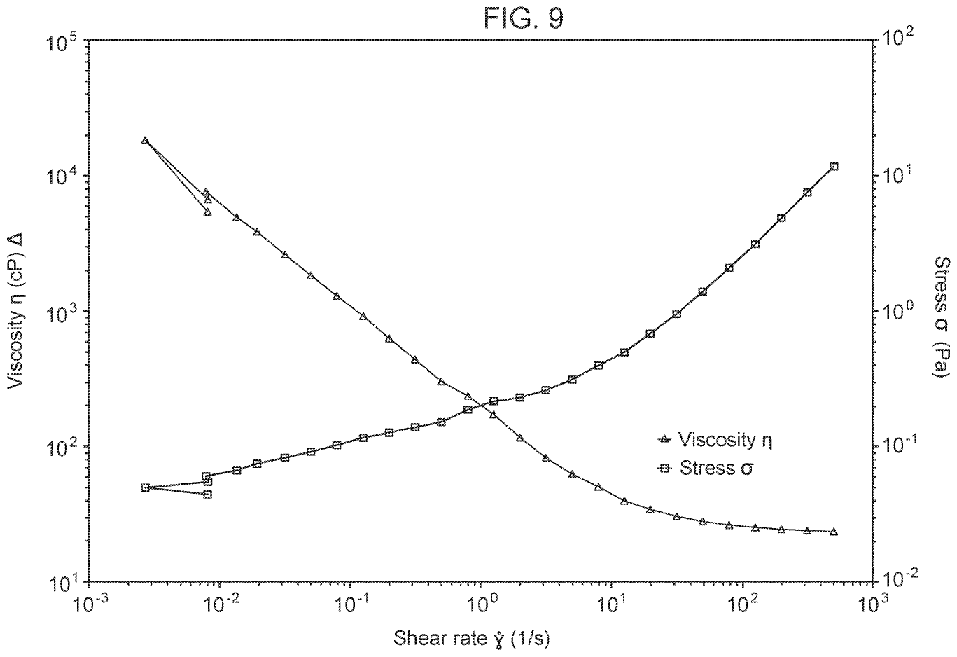

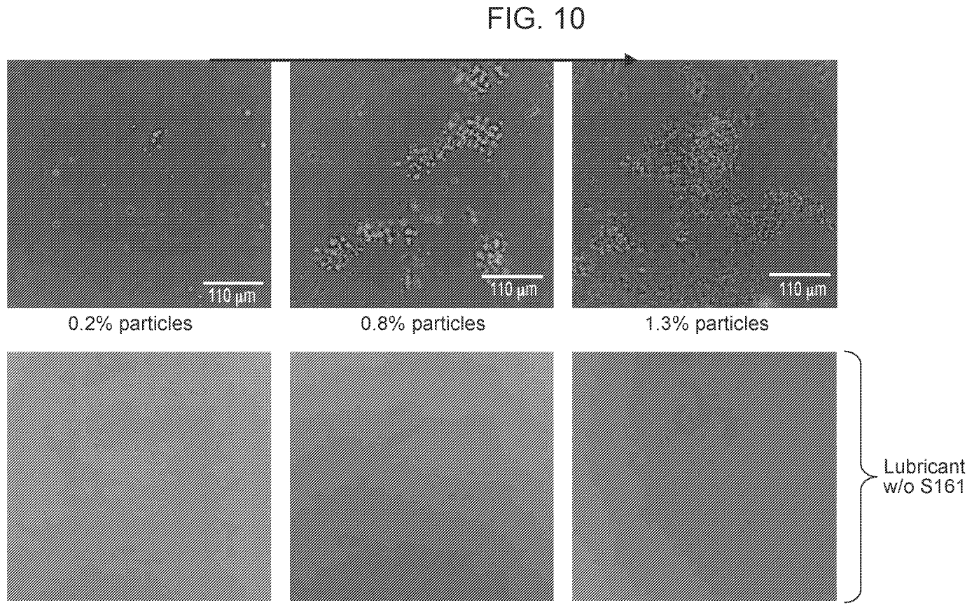

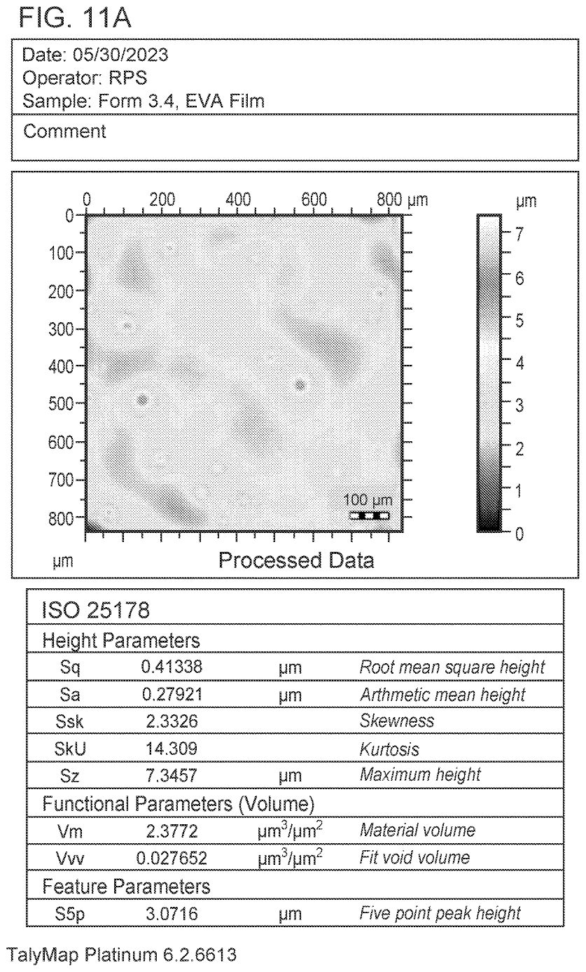

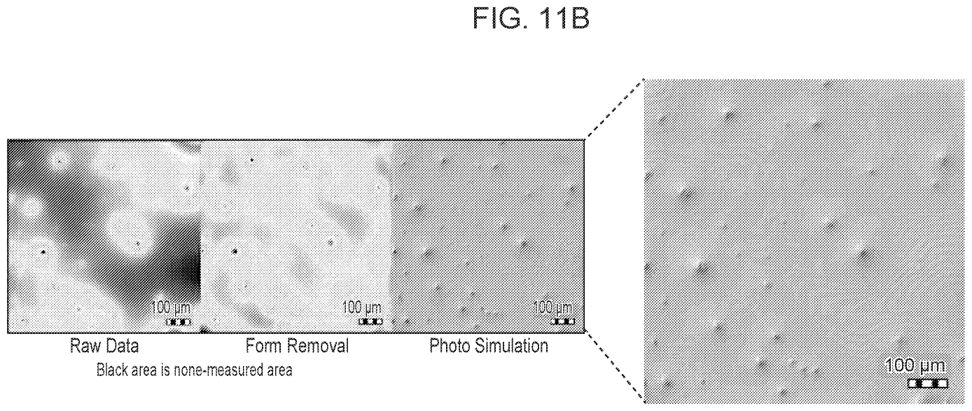

FIG. 9 is a graphical representation of viscosity and stress of the LiquiGlide lubricant as a function of shear rate. As shown, the lubricant is a shear thinning fluid, with the viscosity reducing with increased shear stress. FIG. 10 shows agglomeration of particles in lubricants with various particle concentrations by weight. As shown in the microscopic images, larger weight percentages of particles in the lubricant lead to more agglomeration of the particles. As described above, the size of such “bridging particles” can be selected to adjust the thickness of the lubricant coating on the pouch surface when stool enters the pouch. The particles can prevent the stool from sticking to the side of the pouch. FIGS. 11A-11C display contoured images of lubricants with particles, as compared to lubricants without particles. FIG. 11A depicts an image from the interferometer microscope of an exemplary lubricant with particles. FIG. 11B presents an unprocessed image from the interferometer microscope of an exemplary lubricant with particles, alongside a processed version of this image with form removal applied, and a simulated photograph derived from the image data. FIG. 11C provides three-dimensional views of an interferometer microscope image of both a lubricant with particles and a lubricant without particles. The parameters utilized by the interferometer microscope are detailed in FIG. 11A.

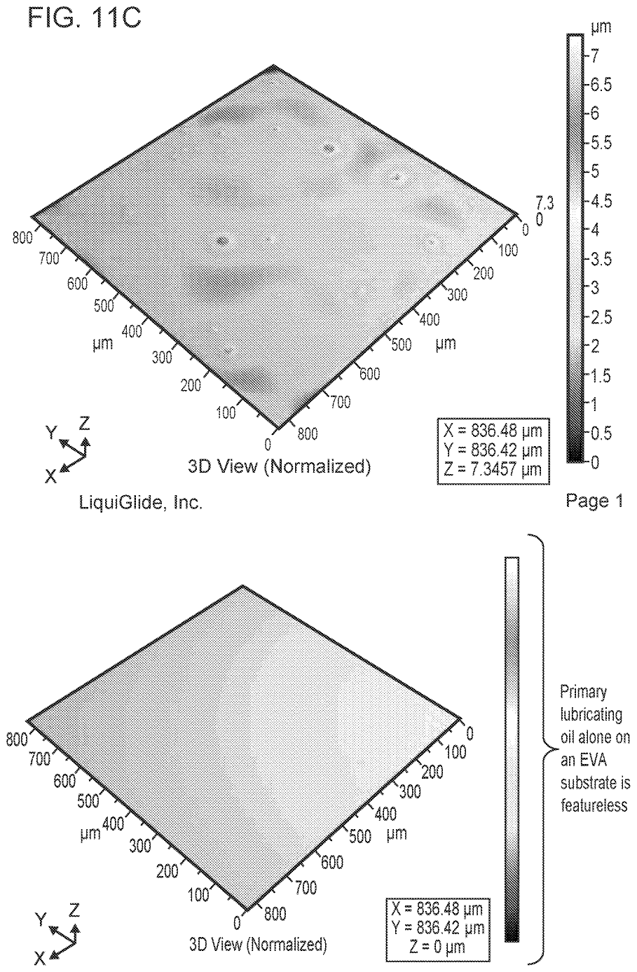

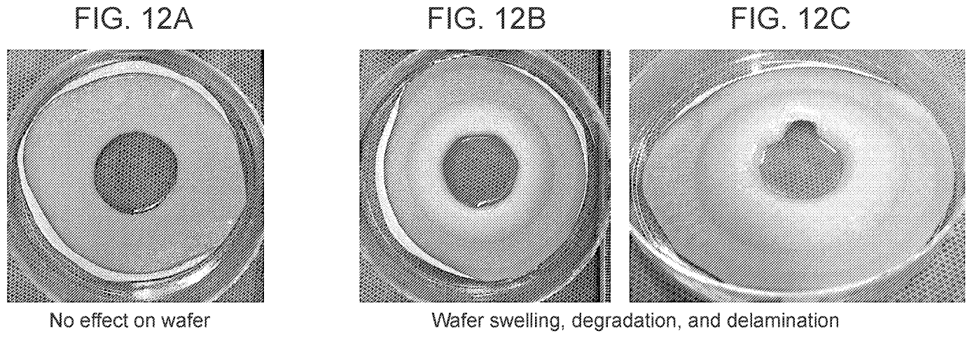

FIGS. 12A-12C show visual representations of a lubricants' effects on an adhesive ostomy wafer. FIG. 12A shows the ostomy wafer stored in LiquiGlide lubricant, while FIGS. 12B and 12C show the ostomy wafer stored in a commercially available lubricant (Esenta™). ConvaTec Esteem® pouch wafers were used, with a stoma opening cut to 30 mm. The wafers adhered to petri dishes and the stoma opening was filled with lubricant. The wafers were stored at 37° C. for 72 hours. Lubricant was replenished as needed. The LiquiGlide lubricant did not require replenishment, while approximately 2.5 mL of Esenta™ lubricant was absorbed by the ostomy wafer every 24 hours. As shown, the ostomy wafer showed little to no degradation after 72 hours in the LiquiGlide lubricant, while the ostomy wafer decomposed significantly in the Esenta™ lubricant.

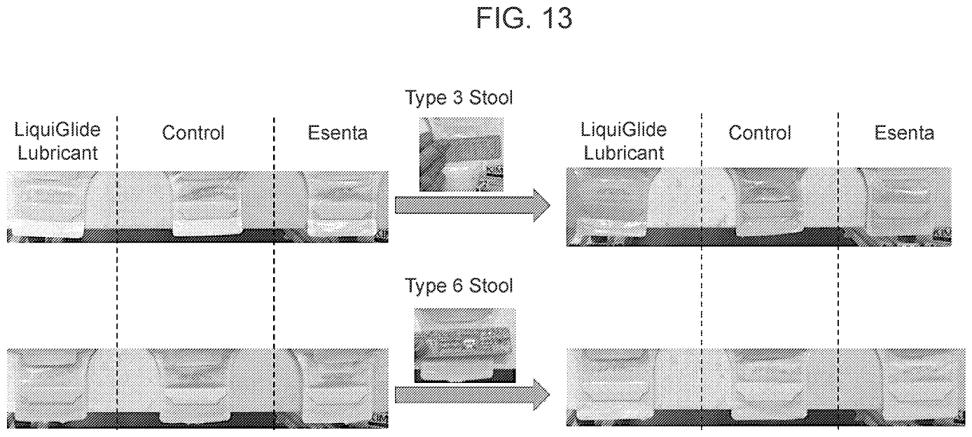

FIG. 13 is a photograph of a pouch tail squeeze method of removing stool from ostomy bags and residual stool mass remaining thereafter. As shown, less waste is present in tail of the pouches coated with LiquiGlide lubricant than with Esenta™ lubricant before the squeeze. After the tail squeeze, the pouches coated with Esenta™ lubricant and control become cleaner with less waste apparent in tail, while the pouch with the LiquiGlide lubricant remains as clean as observed before the squeeze.

Various concepts may be embodied as one or more methods, of which at least one example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments. Put differently, it is to be understood that such features may not necessarily be limited to a particular order of execution, but rather, any number of threads, processes, services, servers, and/or the like that may execute serially, asynchronously, concurrently, in parallel, simultaneously, synchronously, and/or the like in a manner consistent with the disclosure. As such, some of these features may be mutually contradictory, in that they cannot be simultaneously present in a single embodiment. Similarly, some features are applicable to one aspect of the innovations, and inapplicable to others.

In addition, the disclosure may include other innovations not presently described. Applicant reserves all rights in such innovations, including the right to embodiment such innovations, file additional applications, continuations, continuations-in-part, divisionals, and/or the like thereof. As such, it should be understood that advantages, embodiments, examples, functional, features, logical, operational, organizational, structural, topological, and/or other aspects of the disclosure are not to be considered limitations on the disclosure as defined by the embodiments or limitations on equivalents to the embodiments. Depending on the particular desires and/or characteristics of an individual and/or enterprise user, database configuration and/or relational model, data type, data transmission and/or network framework, syntax structure, and/or the like, various embodiments of the technology disclosed herein may be implemented in a manner that enables a great deal of flexibility and customization as described herein.

All definitions, as defined and used herein, should be understood to control over dictionary definitions, definitions in documents incorporated by reference, and/or ordinary meanings of the defined terms.

As used herein, in particular embodiments, the terms “about” or “approximately” when preceding a numerical value indicates the value plus or minus a range of 10%. Where a range of values is provided, it is understood that each intervening value, to the tenth of the unit of the lower limit unless the context clearly dictates otherwise, between the upper and lower limit of that range and any other stated or intervening value in that stated range is encompassed within the disclosure. That the upper and lower limits of these smaller ranges can independently be included in the smaller ranges is also encompassed within the disclosure, subject to any specifically excluded limit in the stated range. Where the stated range includes one or both of the limits, ranges excluding either or both of those included limits are also included in the disclosure.

The phrase “and/or,” as used herein in the specification and in the embodiments, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the embodiments, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the embodiments, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the embodiments, shall have its ordinary meaning as used in the field of patent law.

As used herein in the specification and in the embodiments, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the embodiments, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

While specific embodiments of the present disclosure have been outlined above, many alternatives, modifications, and variations will be apparent to those skilled in the art. Accordingly, the embodiments set forth herein are intended to be illustrative, not limiting. Various changes may be made without departing from the spirit and scope of the disclosure. Where methods and steps described above indicate certain events occurring in a certain order, those of ordinary skill in the art having the benefit of this disclosure would recognize that the ordering of certain steps may be modified and such modification are in accordance with the variations of the invention. Additionally, certain of the steps may be performed concurrently in a parallel process when possible, as well as performed sequentially as described above. The embodiments have been particularly shown and described, but it will be understood that various changes in form and details may be made.

1. An apparatus, comprising:
a container having an inner surface; and
a lubricating liquid coated on the inner surface, the lubricating liquid including:
between about 50 wt % and about 90 wt % of a primary lubricating oil;
between about 0.01 wt % and about 2 wt % of an antioxidant;
between about 0.1 wt % and about 10 wt % of a polymer; and
between about 1 wt % and about 50 wt % of a secondary liquid, the secondary liquid configured to improve the solubility of the polymer in the primary lubricating oil.

2. The apparats of claim 1, wherein the lubricating liquid further includes between about 0.01 wt % and about 2 wt % of a deodorant.

3. The apparatus of claim 1, wherein the container includes an ostomy pouch.

4. The apparatus of claim 1, further comprising:
a contact product disposed in the container and in contact with the lubricating liquid.

5. The apparatus of claim 4, wherein the contact product includes stool.

6. The apparatus of claim 1, wherein the primary lubricating oil includes at least one of: a vegetable oil, a mineral oil, a synthetic liquid, a polyolefin, a hydrogenated polyolefin, an ester, a silicone, a fluorocarbon, or a genetically modified vegetable oil.

7. The apparatus of claim 6, wherein the primary lubricating oil includes a vegetable oil, the vegetable oil including at least one of natural vegetable oil, soybean oil, olive oil, cottonseed oil, a vegetable oil derivative, synthetic vegetable oil, or a genetically modified vegetable oil.

8. The apparatus of claim 1, wherein the antioxidant includes a deodorant.

9. The apparatus of claim 2, wherein the deodorant includes at least one of chlorophyllin copper, copper citrate, copper gluconate, N-soya-N-ethyl morpholinium ethosulfate (Forestall), vegetable protein extracts, sodium caproyl/lauroyl lactylate, sodium decanoyl/dodecanoyl lactylate, triethyl citrate, essential oils, sage oil, pine oil, eucalyptus oil, Melaleuca armillaris, peppermint oil, or lavender oil.

10. The apparatus of claim 1, wherein the secondary liquid includes at least one of a synthetic liquid, a fatty acid, a fatty acid ester, vitamin E, vitamin D, cholesterol, a polyolefin, a hydrogenated polyolefin, an ester, a silicone, or a fluorocarbon.

11. The apparatus of claim 1, wherein the polymer includes at least one of a wax, alginate, gelatin, a polypeptide, a homopolymer with acrylic acid, a copolymer with acrylic acid, acrylic ester, ally acid, ally ester, cellulose, a cellulose derivative, a carboxylic acid, a hydroxyl moiety, vinyl alcohol, ethylene glycol, propylene glycol, or an ester moiety.

12. A lubricating liquid, comprising:
between about 50 wt % and about 90 wt % of a primary lubricating oil;
between about 0.01 wt % and about 2 wt % of an antioxidant;
between about 0.1 wt % and about 10 wt % of a polymer; and
between about 1 wt % and about 50 wt % of a secondary liquid, the secondary liquid configured to improve the solubility of the polymer in the primary lubricating oil.

13. The lubricating liquid of claim 12, further comprising:
between about 0.01 wt % and about 2 wt % of a deodorant.

14. The apparatus of claim 12, wherein the secondary liquid includes at least one of a synthetic liquid, a fatty acid, a fatty acid ester, vitamin E, vitamin D, cholesterol, a polyolefin, a hydrogenated polyolefin, an ester, a silicone, or a fluorocarbon.

15. The lubricating liquid of claim 12, wherein the polymer is dispersed in the lubricating liquid with a particle size ranging from about 5 μm to about 50 μm.

16. The apparatus of claim 12, wherein the primary lubricating oil includes at least one of: a vegetable oil, a vegetable oil derivative, a mineral oil, a synthetic liquid, a polyolefin, a hydrogenated polyolefin, an ester, a silicone, a fluorocarbon, or a genetically modified vegetable oil.
